(19) United States
(12) Patent Application Publication
(10) Pub. No.: US 2026/0103712 A1
(43) Pub. Date: Apr. 16, 2026

(54) ANTI-FIBROTIC MICRORNA COMPOSITION

(71) Applicant: The Council of the Queensland Institute of Medical Research, Herston, Brisbane, Queensland (AU)

(72) Inventors: Grant A. Ramm, Herston Brisbane, Queensland (AU); Berit Genz, Herston Brisbane, Queensland (AU); Rakesh Naduvile Veedu, Perth (AU)

(21) Appl. No.: 19/113,234

(22) PCT Filed: Sep. 20, 2023
(86) PCT No.: PCT/AU2023/050908

§ 371 Date: Mar. 19, 2025

(30) Foreign Application Priority Data
Sep. 20, 2022 (AU) 2022902721

(51) Int. Cl. C12N 15/113

(52) U.S. Cl. CPC C12N 15/1137; C12N 15/113; C12N 15/1136; C12N 2310/141; C12N 2310/315; C12N 2310/321; C12N 2310/322; C12N 2310/333; C12N 2310/3341; C12N 2310/335; C12N 2310/336

(57) ABSTRACT

Provided herein are compositions and methods for the treatment of fibrosis using miR-25 mimics. More particularly, disclosed herein are novel miR mimetics, and methods for attenuating fibrosis progression in a subject, particularly liver fibrosis.

### RELATED APPLICATIONS

This application claims priority to Australian Provisional Application No. 2022902721 entitled “Anti-Fibrotic MicroRNA Composition” filed on 20 Sep. 2022, the contents of which are incorporated herein by reference to their entirety.

### FIELD OF THE INVENTION

This invention relates generally to compositions and methods for the treatment of fibrosis. More particularly, the present invention relates to novel miR mimetics, and methods for attenuating fibrosis progression in a subject.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Hepatic stellate cells (HSCs) are part of the non-parenchymal cell compartment of the liver and act together with macrophages (Kupffer cells), other non-parenchymal liver cells and hepatocytes to support liver function. In the healthy organ, HSCs are quiescent cells that store cytoplasmic vitamin A, regulate sinusoidal blood flow and possess immune cell function (Geerts 2001). Upon tissue injury, pro-fibrotic factors such as transforming growth factor beta (TGF-β) are released from cells including Kupffer cells and HSCs, causing activation and subsequent transdifferentiation of HSCs into myofibroblast-like cells (Friedman 2008). Activated HSCs, in contrast to their quiescent state, express excessive levels of extracellular matrix (ECM) proteins, and are highly contractile (via α-smooth muscle actin (αSMA) expression), proliferative, inflammatory and pro-fibrotic cells. Following chemokine gradients, HSCs migrate towards the site of injury where they secrete fibrillar collagens type I, III and IV, and regulate ECM degradation through altering expression of matrix metalloproteinases (MMPs) and their inhibitors (tissue inhibitors of metalloproteinases (TIMPs)) (Bataller and Brenner 2005). HSC activation is hence considered a primary event in liver fibrogenesis (via increased collagen expression) (Mederacke et al., 2013). The TGF-1 signalling pathway plays a major role in liver fibrosis progression due to its dysregulation in diseased states, inducing HSC proliferation and excessive collagen expression (Dooley et al., 2001). Constitutively active Notch signalling has also been implicated in HSC activation (Villanueva et al., 2012; Xie et al., 2013) with crosstalk between the TGF-β and Notch signalling pathways highlighted as an important mechanism in the progression of liver fibrosis (Bansal et al. 2015; Wang et al., 2017).

MicroRNAs (miRNAs) are important regulators of a range of cellular processes, including proliferation (Ie Sage et al., 2007), differentiation (Yu et al., 2008), protein and gene expression (Eichhorn et al., 2014; Guo et al., 2010). They interact with the RNA-induced silencing complex (RISC) to bind target messenger RNAs (mRNAs) through complementary base pairing to either repress mRNA translation or promote degradation (Bartel 2009; Ha and Kim 2014). MiRNAs have been explored as novel therapeutics due to their stability and availability in body fluids (e.g., serum) (Krauskopf et al., 2017). Importantly, miRNAs have been implicated in the post-transcriptional gene regulation of TGF-3 and Notch signalling pathways (Ichimura et al., 2011; Inui et al., 2010) and are therefore thought to play a crucial role in fibrogenesis.

The inventors previously demonstrated that miR-25-3p (miR-25) was downregulated in serum of children with Cystic Fibrosis accompanied with liver disease (CFLD), including hepatic fibrosis, compared to those who had Cystic Fibrosis and no liver disease, suggesting a protective role for miR-25 in preventing liver fibrosis development (Cook et al., 2015). More recently, the inventors found that miR-25 was endogenously expressed in human and murine HSCs and upregulated during HSC activation in vitro and in vivo (Genz et al., 2019). Using pull-down experiments and target gene sequencing, the inventors identified ADAM-17 and FKBP14, (integral mediators of Notch signalling), as direct targets of miR-25. MiR-25 overexpression in activated HSCs suppressed ADAM-17 and FKBP14 target gene expression, inhibiting Notch-1 receptor cleavage and translocation of the signalling active intracellular domain (NICD1) into the nucleus. Further, miR-25 prevented TGF-β receptor I (TGF-βRI) expression as a target of the Notch signalling pathway, thereby inhibiting TGF-β-induced collagen I expression (Genz et al., 2019). These results emphasised the potential of miR-25 as an anti-fibrotic agent; however, transient transfection of commercially available miR-25 mimetics into HSCs displayed relatively modest effects, with limited efficiency of target gene repression (up to 25%) (Genz et al., 2019). This inspired the design of proprietary miR-25 mimetics to further study the effects of miR-25 on HSC phenotype.

### SUMMARY OF THE INVENTION

The present invention is predicated in part on the discovery that phosphorothioate-linked, and 2′-O-Methyl-modified miR-25 mimetics, significantly increases the protective, anti-fibrotic effects of miR-25 in activated human HSCs. Specifically, downregulation of target genes FKBP14 and ADAM-17 was significantly increased compared to the commercially available mimetic, resulting in the consequent inhibition of TGF-βR1 and TGFβ-induced collagen type 1α1 (COL1 A1) expression. Furthermore, mRNA expression of fibrillar collagens, type I (COL1 A1, COL1A2) and III (COL3A1) was significantly downregulated. Accordingly, the inventors have conceived that the miR-25 proprietary mimetics can be used as a potential novel anti-fibrotic therapeutic for attenuating liver fibrosis progression due to improved efficacy in inhibiting TGF-β-induced fibrillar collagen expression.

In some embodiments, the first strand corresponds to nucleotide residues 52-73 of the mature miR-25 sequence set forth in SEQ ID NO:1 conjugated to two uracil residues at the 3′ termini and having at least one modified nucleotide. In some embodiments, the second strand hybridises at least under low stringency conditions to the first strand and having at least one modified nucleotide.

In some embodiments, the modified nucleotides comprise nucleotides with backbone modification and/or sugar residues. In some embodiments of this type, the backbone modification comprises one or more phosphorothioate, morpholino, methyl phosphonate, amide linkages, or phosphonocarboxylate linkages. In some preferred embodiments, the first two nucleotides at the 5′ termini of the first strand are linked to each other by phosphorothioate linkages. In some preferred embodiments, the last 4 or 5 nucleotides of the 3′ termini of the first strand are linked to each other by phophorothioate linkages. In another embodiment, least one of the 3′ nucleotides of the first strand is a 2′-O-methyl modified nucleotide. In another embodiment, at least one nucleotide of the first strand is a 2′-fluoro nucleotide. In some embodiments, the first strand does not have a modified sugar residue at the second position at both the 5′ and 3′ terminis.

In some embodiments, the backbone modification of the second strand comprises one or more of a phosphorothioate, morpholino, methyl phosphonate, amide, or phosphonocarboxylate linkage. In some preferred embodiments, the first two nucleotides of the 5′ termini of the second strand are linked by a phosphorothioate linkage. In another embodiment, the last seven nucleotides of the 3′ termini of the second strand are linked by phosphorothioate linkages. In another embodiment, the first nucleotide at the 5′ termini of the second strand is a 2′-O-methyl modified nucleotide. In another embodiment, the first seven nucleotides at the 3′ termini of the second strand are 2′-O-methyl modified nucleotides.

In another aspect of the present invention, the miR-25 mimetic compound is presented in Table 1. In some embodiments, the first strand comprises a sequence selected from SEQ ID NO: 2, 4, 9, 11, 12, 13, or 14 and the second strand comprises the sequence set forth in of SEQ ID NO:5. In a preferred embodiment, the first strand comprises the sequence of SEQ ID NO:2 and the second strand comprises the sequence of SEQ ID NO:5. In another preferred embodiment, the first strand comprises the sequence of SEQ ID NO:11 and the second strand comprises the sequence of SEQ ID NO:5. In yet another preferred embodiment, the first strand comprises the sequence of SEQ ID NO:13 and the second strand comprises the sequence of SEQ ID NO:5.

In another aspect, the present invention provides a pharmaceutical composition comprising a miR-25 mimetic compound that comprises, consists, or consists essentially of a first strand comprising a sequence selected from Table 1 or Table 2, and a second strand comprising the sequence set forth in SEQ ID NO: 5; and a pharmaceutically acceptable carrier, excipient and/or diluent.

In still yet another aspect, the present invention provides a method for treating or preventing fibrosis in a subject. By way of an illustrative example, the fibrosis is liver fibrosis.

In some embodiments, the miR-25 compound reduces the expression of COL1A1, COL1A2, COL3A1, COL4A3, COL5A2, COL11A1, FN1, MMP2, CTGF, TGFB2, and/or TGFB3.

In some embodiments, the mir-25 mimetic compound is for the manufacture of a medicament for the therapeutic treatment of fibrosis (e.g., liver fibrosis).

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1. Concentration optimization of novel miR-25 mimetics using FKBP14 target gene downregulation. Novel miR-25 mimetics were titrated from 5 pmol/mL to 50 pmol/mL (Combination 1-8; A-H). Commercial miR-25 (CM) and negative control (-ve) mimetics were used at 20 pmol/mL, as previously described (Genz, B et al. Scientific Reports, 2019). LX-2 cells were transiently transfected with miR-25 mimetics or control (C. elegans non-specific miRNA mimetic) and analysed after 48-hours using qRT-PCR. Optimal target gene downregulation was assessed using FKBP14 relative mRNA expression, normalised to GAPDH and negative control expression. Data are presented as mean±SEM. n=3-4. Data were analysed using one-way ANOVAs with Dunnett's post-hoc analysis (* indicates P<0.05).

FIG. 2. Concentration optimization of novel miR-25 mimetics using FKBP14 target gene downregulation. Novel miR-25 mimetics were titrated from 5 pmol/mL to 40 pmol/mL (Combination 10-16). Negative control (-ve) mimetics were used at 20 pmol/mL, as previously described (Genz, B et al. Scientific Reports, 2019). LX-2 cells were transiently transfected with miR-25 mimetics or control (C. elegans non-specific miRNA mimetic) and analysed after 48-hours using qRT-PCR. Optimal target gene downregulation was assessed using FKBP14 relative mRNA expression, normalised to GAPDH and negative control expression. Data are presented as mean±SEM. n=3. Data were analysed using one-way ANOVAs with Dunnett's post-hoc analysis (* indicates P<0.05).

FIG. 3. Analysis of C3 miR-25 mimetic effect on FKBP14 and ADAM-17 mRNA and protein expression. Optimised concentrations of Combination 3 (C3; 5, 20, 40 pmol/mL), commercial miR-25 mimetic (CM; 20 and 40 pmol/mL) and negative control (-ve; non-specific C. elegans miRNA mimetic; 20 pmol/mL) were transfected into LX-2 cells. (A-B) mRNA expression of previously described 20 key target genes of miR-25, FKBP14 and ADAM-17, were analysed using qRT-PCR, 48-hours after transfection. (C-D) Protein expression was analysed using Western blots, 72-hours after transfection. Data are presented as mean±SEM. n=3-12. Data were analysed using one-way ANOVAs with Dunnett's post-hoc analysis (* indicates P<0.05).

FIG. 4. 48-hour protein expression analysis of miRNA-25 targets. Protein expression of miRNA-25 targets, FKBP14 (A) and ADAM-17 (B), and TGFBR1 (C) were analysed using Western blots, 48-hours after transfection with C3 vs CM vs negative control. Data are presented as mean±SEM. n=7-18. Data were analysed using one-way ANOVAs with Dunnett's post-hoc analysis.

FIG. 5. Analysis of C13 and C15 miR-25 mimetic on FKBP14 and ADAM-17 mRNA and protein expression. Optimised concentrations of Combination 13 (C13) and Combination 15 (C15; 5, 20, 40 pmol/mL), and negative control (-ve; non-specific C. elegans miRNA mimetic; 20 pmol/mL) were transfected into LX-2 cells. mRNA expression of previously described key target genes of miR-25, FKBP14 (A, C) and ADAM-17 (B, D), were analysed using qRT-PCR, 48-hours after transfection. Data are presented as mean±SEM. n=3-5. Data were analysed using one-way ANOVAs with Dunnett's post-hoc analysis (* indicates P<0.05).

FIG. 6. Analysis of C3 miR-25 mimetic effect on secretion, mRNA and protein expression of Collagen type 1α1. Optimised concentrations of C3 (5, 20, 40 pmol/mL), commercial miR-25 mimetic (CM; 20 and 40 pmol/mL) and negative control (-ve; non-specific C. elegans miRNA mimetic; 20 pmol/mL) were transfected into LX-2 cells. (A) mRNA expression of fibrillar Collagen 1α1 was analysed using qRT-PCR 48-hours after transfection. (B) Protein expression of fibrillar Collagen 1α1 was analysed using Western blots, 72-hours after transfection. (C) Cell supernatant was collected from transfected LX-2 cells, 72-hours after transfection. Collagen secretion was analysed using the Abcam Human Pro-Collagen I alpha 1 ELISA Kit. (D) A TGF-β stimulation assay was performed by stimulating transfected LX-2 cells 24-hours after transfection with recombinant human TGF-β (10 ng/mL) or RNase-free water (control treatment (Ctrl)) for 24-hours. Relative expression of Collagen-1α1 and α-SMA mRNA was analysed using qRT-PCR. Data are presented as mean±SEM. n=3-15. Data were analysed using one-way ANOVAs with Dunnett's post-hoc analysis (* indicates P<0.05, ** indicates P<0.01, *** indicates P<0.001, **** indicates P<0.0001).

FIG. 7. Analysis of C13 and C15 miR-25 mimetics effect on secretion, mRNA and protein expression of Collagen type 1α1 and 1α2. Optimised concentrations of Combination 13 and 15 (5, 20, 40 pmol/mL), and negative control (-ve; non-specific C. elegans miRNA mimetic; 20 pmol/mL) were transfected into LX-2 cells. mRNA expression of fibrillar Collagen 1 al (A, C) and 1α2 (B, D) were analysed using qRT-PCR 48-hours after transfection. Data are presented as mean±SEM. n=3-5. Data were analysed using one-way ANOVAs with Dunnett's post-hoc analysis (* indicates P<0.05, ** indicates P<0.01, *** indicates P<0.001, **** indicates P<0.0001).

FIG. 8. Effects of C3 miR-25 mimetic on fibrillar collagen (secretion), mRNA and protein expression. Optimised concentrations of C3 (5, 20, 40 pmol/mL), commercial miR-25 mimetic (CM; 20 and 40 pmol/mL) and negative control (-ve; non-specific C. elegans miRNA mimetic; 20 pmol/mL) were transfected into LX-2 cells. (A) mRNA expression of fibrillar collagens type 1α2 and 3α1 were analysed using qRT-PCR, 48-hours after transfection. (C-D) Protein expression of fibrillar Collagens 1α2 and 3α1 were analysed using Western blots, 72-hours after transfection. Data are presented as mean±SEM. n=3-16. Data were analysed using one-way ANOVAs with Dunnett's post-hoc analysis (* indicates P<0.05, ** indicates P<0.01, *** indicates P<0.001, **** indicates P<0.0001).

FIG. 9. Target gene analysis of gelatinous collagen type IV using C3 miR-25 mimetic. Optimised concentrations of C3 (5, 20, 40 pmol/mL), commercial miR-25 mimetic (CM; 20 pmol/mL) and negative control (-ve; non-specific C. elegans miRNA mimetic; 20 pmol/mL) were transfected into LX-2 cells. mRNA expression of gelatinous collagen IV was analysed using qRT-PCR, 48-hours after transfection. Data are presented as mean±SEM. n=6-15. Data were analysed using one-way ANOVAs with Dunnett's post-hoc analysis (* indicates p<0.05, ** indicates p<0.01).

FIG. 10. Effects of C3 miR-25 mimetic on components of the TGF-β signalling pathway. Optimised concentrations of C3 (5, 20, 40 pmol/mL), commercial miR-25 mimetic (CM; 20 and 40 pmol/mL) and negative control (-ve; non-specific C. elegans miRNA mimetic; 20 pmol/mL) were transfected into LX-2 cells. (A) mRNA expression of TGF-β receptor 1 was analysed using qRT-PCR, 48-hours after transfection. (B) Protein expression of TGF-β receptor 1 was analysed using Western blots, 72-hours after transfection. (C-F) mRNA expression of other components of TGF-β signalling were analysed using qRT-PCR, 48-hours after transfection. Data are presented as mean±SEM. n=2-15. Data were analysed using one-way ANOVAs with Dunnett's post-hoc analysis (* indicates p<0.05, ** indicates p<0.01, *** indicates p<0.001, **** indicates p<0.0001).

FIG. 11. Effects of C13 and C15 miR-25 mimetic on fibrillar collagen (secretion) and TGF-β signalling pathway, mRNA and protein expression. Optimised concentrations of C13 and C15 (5, 20, 40 pmol/mL), and negative control (-ve; non-specific C. elegans miRNA mimetic; 20 pmol/mL) were transfected into LX-2 cells. mRNA expression of fibrillar collagens type 3α1 and TGF-β receptor 1 were analysed using qRT-PCR, 48-hours after transfection. Data are presented as mean±SEM. n=3-5. Data were analysed using one-way ANOVAs with Dunnett's post-hoc analysis (* indicates P<0.05, ** indicates P<0.01, *** indicates P<0.001, **** indicates P<0.0001).

FIG. 12. Target gene analysis of HSC ECM modulating factors using C3 miR-25 mimetic. Optimised concentrations of C3 (5, 20, 40 pmol/mL), commercial miR-25 mimetic (CM; 20 pmol/mL) and negative control (-ve; non-specific C. elegans miRNA mimetic; 20 pmol/mL) were transfected into LX-2 cells. mRNA expression of collagen modulating factors were analysed using qRT-PCR, 48-hours after transfection. Data are presented as mean±SEM. n=6-16. Data were analysed using one-way ANOVAs with Dunnett's post-hoc analysis (* indicates p<0.05, ** indicates p<0.01, * indicates p<0.001).

FIG. 13. Effect of C3 mimetic on markers of HSC activation. LX-2 cells were transfected with C3 (20 and 40 pmol/mL), commercial miR-25 mimetic (CM; 20 and 40 pmol/mL) and a non-specific C. elegans miRNA mimetic as negative control (-ve; 40 pmol/mL). (A) Cell migration analysis. A wound was implemented into the confluent cell layer using a scratch wound maker. Wound width (μm) was measured every 2 hours for 24-hours using the IncuCyte Zoom live cell analysis system (left panel). Wound width after 24 h is presented in the right panel. (B) Cell Proliferation analysis. Cells were incubated in the IncuCyte Zoom live cell analysis system for up to 7 days and confluency was measured every 3 hours (left panel). Growth rate over time was calculated as growth constants (K; right panel). (C) Cell contractility analysis. Transfected cells were reseeded onto a collagen lattice and contraction was stimulated by adding endothelin-1 (10 nM), 48-hours after transfection. Collagen matrix shrinkage was measured at 0.5, 1, 2.5 and 6 hours after addition of endothelin-1 (left panel). Collagen area after 6 h is shown in the right panel. Data are presented as mean±SEM. n=5-16. Data were analysed using one-way ANOVAs with Dunnett's post-hoc analysis.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

As used herein, the indefinite articles “a” and “an” are used here to refer to or encompass singular or plural elements or features and should not be taken as meaning or defining “one” or a “single” element or feature. For example, “a” protein includes one protein, one or more proteins or a plurality of proteins.

The term “about” as used herein refers to the usual error range for the respective value readily known to the skilled person in this technical field. Reference to “about” a value or parameter herein includes (and describes) embodiments that are directed to that value or parameter per se.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc. The term “agent” is not to be construed narrowly but extends to small molecules, bicyclic peptide mimetics such as peptides, polypeptides and proteins as well as compositions comprising them and genetic molecules such as RNA, DNA and mimetics and chemical analogues thereof as well as cellular agents.

As used herein, “and/or” refers to and encompasses any and all possible combinations of one or more of the associated listed items, as well as the lack of combinations when interpreted in the alternative (or).

By “coding sequence” is meant any nucleic acid sequence that contributes to the code for the polypeptide product of a gene or for the final mRNA product of a gene (e.g., the mRNA product of a gene following splicing). By contrast, the term “non-coding sequence” refers to any nucleic acid sequence that does not contribute to the code for the polypeptide product of a gene or for the final mRNA product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

“Complementary” means each nucleobase of an oligonucleotide is capable of pairing with a nucleobase at each corresponding position in a target nucleic acid. In certain embodiments, an oligonucleotide is fully complementary to a microRNA, i.e., each nucleobase of the oligonucleotide is complementary to a nucleobase at a corresponding position in the microRNA. In certain embodiments, an oligonucleotide wherein each nucleobase has complementarity to a nucleobase within a region of a microRNA stem-loop sequence is fully complementary to the microRNA stem-loop sequence.

By “corresponds to” or “corresponding to” is meant a nucleotide sequence that displays substantial sequence similarity or identity to a reference amino acid sequence. In general, the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

By “derivative” is meant a molecule, such as a nucleotide sequence, that has been derived from the basic molecule by modification, for example by conjugation or complexing with other chemical moieties or by post-translational modification techniques as would be understood in the art. The term “derivative” also includes within its scope alterations that have been made to a parent sequence including additions or deletions that provide for functionally equivalent molecules.

An “effective amount” is at least the minimum amount required to affect a measurable improvement or prevention of a particular disorder. An effective amount herein may vary according to factors such as the disease state, age, sex, and weight of the patient, and the ability of a polynucleotide to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioural symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

“Fibrosis” means the formation or development of excess fibrous connective tissue in an organ or tissue. In certain embodiments, fibrosis occurs as a reparative or reactive process. In certain embodiments, fibrosis occurs in response to damage or injury. The term “fibrosis” is to be understood as the formation or development of excess fibrous connective tissue in an organ or tissue as a reparative or reactive process, as opposed to a formation or fibrous tissue as anormal constituent of an organ or tissue.

The term “high”, as used herein, refers to a measure that is greater than normal, greater than a standard such as a predetermined measure or a subgroup measure or that is relatively greater than another subgroup measure. A normal measure may be determined according to any method available to one skilled in the art. The term “high” may also refer to a measure that is equal to or greater than a predetermined measure, such as a predetermined cut-off. If a subject is not “high” for a particular marker, it is “low” for that marker. In general, the cut-off used for determining whether a subject is “high” or “low” should be selected such that the division becomes clinically relevant.

“Hybridization” is used herein to denote the pairing of complementary nucleotide sequences to produce a DNA-DNA hybrid or a DNA-RNA hybrid or an RNA-RNA hybrid.

Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T/U and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The term “inhibitor” as used herein refers to an agent that decreases or inhibits at least one function or biological activity of a target molecule.

The term “locked nucleic acid (LNA)” refers to a substituted and conformationally restricted sugar moiety comprising a methylene bridge between the 4′ and 2′ furanose ring atoms.

Throughout the disclosure, the term “microRNA mimetic compound” may be used interchangeable with the terms “promiR-25”, “miR-25 agonist”, “microRNA agonist”, “microRNA mimetic”, “miRNA mimetic”, or “miR-25 mimetic”, means an endogenous non-coding RNA between 18 and 25 nucleobases in length, which is the product of cleavage of a pre-microRNA by the enzyme Dicer. Example of mature microRNA are found in the microRNA database known as miRbase (http://microrna.sanger.ac.uk/). In certain embodiments, microRNA is abbreviated as “miR”. The term “first strand” may be used interchangeably with the term “antisense strand” or “guide strand”; and the term “second strand” may be used interchangeably with the term “sense strand” or “passenger strand”.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

As used herein, the terms “peptide”, “polypeptide” and “protein” are used interchangeably to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally-occurring amino acid, as well as to naturally-occurring amino acid polymers. These terms do not exclude modifications, for example, glycosylation, acetylation, phosphorylation and the like. Soluble forms of the subject peptides are particularly useful. Included within the definition are, for example, peptides containing one or more analogues of an amino acid including, for example, unnatural amino acids or polypeptides with substituted linkages.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, colouring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

The term “phosphorothioate linkage” refers to a linkage between nucleosides where one of the non-bridging oxygen atoms is replaced with a sulfur atom.

As used herein, the terms “prevent”, “prevented” or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, fatigue, etc. In another embodiment, the reduction may be determined objectively. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, the terms “salts” and “prodrugs” include any pharmaceutically acceptable salt, ester, hydrate or any other compound which, upon administration to the recipient, is capable of providing (directly or indirectly) a miR-25 mimetic of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs can be carried out by methods known in the art. For example, metal salts can be prepared by reaction of a compound of the invention with a metal hydroxide.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., faeces), tears, sweat, sebum, nipple aspirate, ductal lavage, tumour exudates, synovial fluid, ascitic fluid, peritoneal fluid, amniotic fluid, cerebrospinal fluid, lymph, fine needle aspirate, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC).

The term “first strand” may be used interchangeably with the terms “antisense strand” or “guide strand”; and the term “second strand” may be used interchangeably with the term “sense strand” or “passenger strand”.

“Stringency” as used herein, refers to temperature and ionic strength conditions, and presence or absence of certain organic solvents and/or detergents during hybridisation. The higher the stringency, the higher will be the required level of complementarity between hybridising nucleotide sequences.

“Stringent conditions” designates those conditions under which only nucleic acid having a high frequency of complementary bases will hybridise.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimin) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In some embodiments, the subject is a mammal. In other embodiments, the subject is a human.

As used herein, the terms “treatment”, “treating” and the like refer to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a fibrosis disorder are mitigated or eliminated, including, but are not limited to, reducing the proliferation of (or destroying) fibrosis, decreasing symptoms resulting from the disease, increasing the quality of life of those suffering from the disease, decreasing the dose of other medications required to treat the disease, and/or prolonging survival of individuals.

The term “2′-O-fluoro” means a sugar having a fluoro modification at the 2′ position.

The term ‘2′-O-methyl” refers to a sugar having a O-methyl modification at the 2′ position.

Each embodiment described herein is to be applied mutatis mutandis to each and every embodiment unless specifically stated otherwise.

### 2. Compositions

The present invention is predicated in part on the discovery that phosphorothioate-linked, and 2′-O-methyl-modified miR-25 mimetics significantly increase the protective, anti-fibrotic effects of miR-25 in activated human HSCs. Specifically, downregulation of target genes FKBP14 and ADAM-17 was significantly increased compared to the commercially available mimetic, resulting in the consequent inhibition of TGF-βR1 and TGFβ-induced collagen type 1α1 (COL1A1) expression. Furthermore, mRNA expression of fibrillar collagens, type I (COL1A1, COL1A2) and III (COL3A1) was significantly downregulated. Accordingly, the present invention provides a miR-25 novel mimetic that can be used as a novel anti-fibrotic therapeutic for controlling fibrosis progression due to its improved efficacy in inhibiting TGF-β-induced fibrillar collagen expression.

2.1 miR-25 Mimetic

MicroRNAs (miRNA) are a class of non-coding RNAs, belonging to a class of regulatory molecules found in plants and animals that control gene expression by binding to complementary sites on target messenger RNA (mRNA) transcripts. miRNAs are generated from larger RNA precursors (termed pri-miRNAs) that are processed in the nucleus into approximately 70 nucleotide pre-miRNAs, which fold into imperfect stem-loop structures. The pre-miR-NAs undergo an additional processing step within the cytoplasm where mature miRNAs of 18-25 nucleotides in length are excised from one side of the pre-miRNA hairpin by RNase III enzyme Dicer.

miRNAs have been shown to regulate gene expression in two ways. First, miRNAs that bind to protein-coding mRNA sequences that are exactly complementary to miRNA induce the RNA-mediated interference (RNAi) pathway. Messenger RNA targets are cleaved by ribonucleases in the RISC complex. In the second mechanism, miRNAs that bind to imperfect complementary sites on messenger RNA transcripts direct gene regulation at the post transcriptional level but do not cleave their mRNA targets. miRNAs identified in both plants and animals use this mechanism to exert translational control over their gene targets.

As used herein, the “microRNA” (miRNA or miR) includes mature single stranded miRNAs precursor miRNAs (pre-miR), and variants thereof, which may be naturally occurring. In some instances, the term “miRNA” also includes primary miRNA transcripts and duplex miRNAs. Unless otherwise noted, when used herein, the name of a specific miRNA refers to the mature miRNA of a precursor miRNA. For example, miR-25 refers to a mature miRNA sequence derived from pre-miR-25.

The native mature pri-miR-25 sequence (hsa-miR-25-3p miRBase Accession No: MIMAT0000081) is set forth below.

[SEQ ID NO: 1]
GGCCAGUGUUGAGAGGCGGAGACUUGGGCAAUUGCUGGACGC
UGCCCUGGGCAUUGCACUUGUCUCGGUCUGACAGUGCCGGCC.

The miR-25 mimetics of the invention comprise a first strand and a second strand, wherein the first strand comprises, consists, or consists essentially of a mature miR-25 sequence (i.e., CAUUGCACUUGUCUCGGUCUGA [SEQ ID NO: 3]) and the second strand comprises a sequence that is substantially complementary to the first strand and has at least one modified nucleotide.

In some embodiments, the nucleotide sequence set forth in SEQ ID NO:3 or a fragment, variant or derivative thereof, two nucleotide residues overhanging at the 3′ terminal and does not contain a modification at both the second nucleotide positions at the 5′ terminal and 3′ terminal and the second strand comprising a sequence that is substantially complementary to the first strand, comprising a modification at the first residue of the 3′ terminal and a modified linker between each of the last seven nucleotides of the 5′ terminal. The term “modified nucleotide” means a nucleotide where the nucleobase and/or sugar moiety is modified relative to unmodified nucleotides.

In some embodiments, the first strand of the microRNA mimetic compound comprises from about 24 nucleotides comprising a sequence of mature miR-25 and the second strand comprises around 24 nucleotides comprising a sequence that is partially, substantially, or fully complementary to the first strand. In various embodiments, the first strand may comprise about 22, 23, 24, 25, or 26 nucleotides and the second strand may comprise about 22, 23, 24, 25, or 26 nucleotides.

The nucleotides that form the first strand of the microRNA mimetic compounds may comprise ribonucleotides, deoxyribonucleotides, modified nucleotides, and combinations thereof. In certain embodiments, the first strand and the second strand of the microRNA mimetic comprise ribonucleotides and/or modified ribonucleotides. The term “modified nucleotide” means a nucleotide where the nucleobase and/or the sugar moiety is modified relative to unmodified nucleotides.

In certain embodiments, the microRNA mimetic compounds have a first strand or an antisense strand, whose sequence is identical to all or part of a mature miR-25 sequence, and a second strand or a sense strand whose sequence is about 70% to about 100% complementary to the sequence of the first strand. In some embodiments, the first strand of the miRNA mimetic compound is at least about 75, 80, 85, 90, 95, or 100% identical, including all integers therebetween, to the entire sequence of a mature, naturally occurring miR-25 sequence. In certain embodiments, the first strand is about or is at least about 90, 91, 92, 93, 94, 95, 96, 97, 98, 99, or 100% identical to the sequence of a mature, naturally occurring miRNA, such as the human, mouse, or rat miR-25 sequence. Alternatively, the first strand may comprise 20, 21, 22, or 23 nucleotide positions in common with a mature, naturally-occurring miRNA as compared by sequence alignment algorithms and methods well known in the art.

It is understood that the sequence of the first strand is considered to be identical to the sequence of a mature miR-25 even if the first strand includes a modified nucleotide instead of a naturally-occurring nucleotide. For example, if a mature naturally-occurring miRNA sequence comprises a cytidine nucleotide at a specific position, the first strand of the mimetic compound may comprise a modified cytidine nucleotide, such as 2′-fluoro-cytiding, at the corresponding position or if a mature, naturally-occurring miRNA sequence comprises a uridine nucleotide at a specific position, the miRNA region of the first strand of the mimetic compound may comprise a modified uridine nucleotide, such as 2′-fluoro-uridine, 2′-O-methyl-uridine, 5′-fluorouracil, or 4-thiouracil at the corresponding position. Thus, as long as the modified nucleotide has the same base-pairing capability as the nucleotide present in the mature, naturally-occurring miRNA sequence, the sequence of the first strand is considered to be identical to the mature, naturally-occurring miRNA sequence. In some embodiments, the first strand may have a 5′-terminal monophosphate. In some other embodiments, the first strand does not contain a 5′-terminal monophosphate.

In some embodiments, the second strand of the microRNA mimetic compounds is partially complementary to the sequence of the first strand. For example, the sequence of the second strand is at least about 70, 75, 80, 85, 90, 95, or 99% including all integers therebetween, complementary to the sequence of the first strand. In yet some other embodiments, the sequence of the second strand may be fully complementary to the first strand. In certain embodiments, about 19, 20, 21, 22, or 23 nucleotides of the complementary region of the second strand may be complementary to the first strand.

In some embodiments, the second strand comprises about 1, 2, 3, 4, 5, or 6 mismatches relative to the first strand. That is, up to 1, 2, 3, 4, 5, or 6 nucleotides between the first strand and the second strand may not be complementary. In one embodiment, the mismatches are not consecutive and are distributed throughout the second strand. In another embodiment, the mismatches are consecutive and may create a bulge. In some embodiments, the second strand contains one, two, or three mismatches relative to the first strand.

In some embodiments, the first and/or the second strand of the mimetic compound may comprise an overhang on the 5′ or 3′ end of the strands. In certain embodiments, the first strand comprises a 3′ overhang, i.e., a single-stranded region that extends beyond the duplex region, relative to the second strand. The 3′ overhang of the first strand may range from about one nucleotide to about four nucleotides. In certain embodiments, the 3′ overhang of the first strand may comprise 1 or 2 nucleotides. In some embodiments, the nucleotides comprising the 3′ overhang in the first strand are linked by phosphorothioate linkages. The nucleotides comprising the 3′ overhang in the first strand may include ribonucleotides, deoxyribonucleotides, modified nucleotides, or combinations thereof. In certain embodiments, the 3′ overhang of the first strand comprises two uridine nucleotides linked through a phosphorothioate linkage. In some embodiments, the first strand may not contain an overhang.

In certain embodiments, the second strand comprises a 3′ overhang, i.e., a single-stranded region that extends beyond the duplex region, relative to the first strand. The 3′ overhang of the second strand may range from about one nucleotide to about four nucleotides. In certain embodiments, the 3′ overhang of the second strand may comprise 1 or 2 nucleotides. In some embodiments, the nucleotides comprising the 3′ overhang in the second strand are linked by phosphorothioate linkages. The nucleotides comprising the 3′ overhang in the second strand may include ribonucleotides, deoxyribonucleotides, modified nucleotides, or combinations thereof. In certain embodiments, the 3′ overhang of the second strand comprises two 2′-O-methyl-uridine nucleotides linked through a phosphorothioate linkage. In some embodiments, the second strand may not contain an overhang.

In some embodiments, the nucleotides in the second/sense strand of the miR-25 mimetics of the invention are linked by phosphodiester linkages except for the last five nucleotides at the 3′ end which are linked to each other via phosphorothioate linkages. In some embodiments, the nucleotides in the first/antisense strand of the miR-25 mimetics of the invention are linked by phosphodiester linkages except for the last two or three nucleotides at the 3′ end which are linked to each other via phosphorothioate linkages.

In various embodiments, miR-25 mimics of the present invention comprise modified nucleotides. For instance, in some embodiments the first strand of the mimic comprises one or more 2′-O-methyl modified nucleotides. In some of the same embodiments and some other embodiments the first strand comprises one or more 2′-fluoro nucleotides. In some preferred embodiments, the first strand may not include any modified nucleotides.

In some embodiments, the second strand comprises one or more 2′-O-methyl modified nucleotides. In some preferred embodiments of this type, the last seven nucleotides at the 3′ end are 2′-O-methyl modified nucleotides.

In some embodiments, the first strand does not contain a modified nucleotide at both the second positions at the 5′ terminal and 3′ terminal ends. In this regard, the first strand may comprise a modified nucleotide at the second position from the 5′ terminal end. Alternatively, the first strand may comprise a modified nucleotide at the second position from the 3′ terminal end.

In various embodiments, miR-25 mimics according to the present invention comprise first and second strands listed in the Tables 1 and 2 below. Definitions of the modifications are presented in Table 3. These miR-25 mimetic compounds are useful for regulating the expression of extracellular matrix genes in a cell and treating associated conditions, such as fibrosis.

TABLE 1
GROUP 1 MIR-25 MIMETIC STRAND SEQUENCES

| | |
|---|---|
| | SEQ |
| | ID |
| Sequence (5′ to 3′) | NO: |

Second/sense/passenger strand

| | |
|---|---|
| 5′-mU*rC.rA.rG.rA.rC.rC.rG.rA.rG.rA.rC. | 5 |
| rA.rA.rG.rU.rG.mC*mA*mA*mU*mG*mU*mU-3′ | |

First/antisense/guide strands

| | |
|---|---|
| 5′-rC*rA.rU.rU.rG.rC.rA.rC.rU.rU.rG.rU. | 2 |
| rC.rU.rC.rG.rG.rU.rC.rU*rG*rA*rU*rU-3′ | |
| 5′-rC*rA.rU.rU.rG.rC.rA.rC.rU.rU.rG.rU. | 14 |
| rC.rU.rC.rG.rG.mU.rC.mU.rG*rA*rU*rU-3′ | |
| 5′-rC*rA.fU.rU.rG.fC.rA.fC.rU.rU.rG.rU. | 16 |
| rC.rU.rC.rG.rG.mU.rC.mU.rG*rA*rU*rU-3′ | |

TABLE 2
GROUP 2 MIR-25 MIMETIC STRAND SEQUENCES

| | |
|---|---|
| | SEQ |
| | ID |
| Sequence (5′ to 3′) | NO: |

Second/sense/passenger strand

| | |
|---|---|
| 5′-mU*rC.rA.rG.rA.rC.rC.rG.rA.rG.rA.rC. | 5 |
| rA.rA.rG.rU.rG.mC*mA*mA*mU*mG*mU*mU-3′ | |

First/antisense/guide strands

| | |
|---|---|
| 5′-rC*rA.rU.rU.rG.rC.rA.rC.rU.rU.rG.rU. | 4 |
| rC.rU.rC.rG.rG.mU.mC.mU*mG*mA*mU*mU-3′ | |
| 5′-rC*mA.rU.rU.rG.rC.rA.rC.rU.rU.rG.rU. | 11 |
| rC.rU.rC.rG.rG.rU.rC.rU*rG*rA*rU*rU-3′ | |
| 5′-rC*rA.rU.rU.rG.rC.rA.rC.rU.rU.rG.rU. | 12 |
| rC.rU.rC.rG.rG.rU.rC.rU*rG*rA*mU*rU-3′ | |
| 5′-rC*rA.fU.rU.rG.fC.rA.fC.rU.rU.rG.rU.′ | 15 |
| rC.rU.rC.rG.rG.rU.rC.rU*rG*rA*rU*rU-3 | |
| 5′-rC*rA.fU.rU.rG.fC.rA.fC.rU.rU.rG.rU. | 17 |
| rC.rU.rC.rG.rG.fU.rC.fU*rG*rA*rU*rU-3′ | |

TABLE 3
ABBREVIATIONS

| Nucleotide unit or | |
|---|---|
| modification | Abbreviation |
| ribo A | rA |
| ribo G | rG |
| ribo C | rC |
| ribo U | rU |
| 2′-O-Methyl A | mA |
| 2′-O-Methyl G | mG |
| 2′-O-Methyl C | mC |
| 2′-O-Methyl U | mU |
| 2′-Fluoro A | fA |
| 2′-Fluoro G | fG |
| 2′-Fluoro C | fC |
| 2′-Fluoro U | fU |
| LNA nucleotide | LT |
| modification | |
| Phosphorothioate | * |
| linkages | |

In certain embodiments, a miR-25 mimic comprises a first strand comprising SEQ ID NO: 2 and a second strand comprising SEQ ID NO: 5. In other embodiments, a miR-25 mimic comprises a first strand comprising SEQ ID NO: 14 and a second strand comprising SEQ ID NO: 5. In yet other embodiments, a miR-25 mimic comprises a first strand comprising SEQ ID NO: 16 and a second strand comprising SEQ ID NO: 5.

In some other embodiments, a miR-25 mimic comprises a first strand comprising SEQ ID NO: 4 and a second strand comprising SEQ ID NO: 5. In yet other embodiments, the miR-25 mimic may comprise a first strand comprising SEQ ID NO: 11 and a second strand comprising SEQ ID NO: 5. In yet other embodiments, the miR-25 mimic may comprise a first strand comprising SEQ ID NO: 12 and a second strand comprising SEQ ID NO: 5. In still yet other embodiments, the miR-25 mimic comprises a first strand comprising SEQ ID NO: 15 and a second strand comprising SEQ ID NO: 5. In yet other embodiments, a miR-25 mimic comprises a first strand comprising SEQ ID NO: 17 and a second strand comprising SEQ ID NO: 5.

The modifications that may be used in the miR-25 mimetic compounds of the disclosure can include nucleotides with a base modification or substitution. The natural or unmodified bases in RNA are the purine bases adenine (A) and guanine (G), and the pyrimidine bases cytosine (C) and uracil (U) (DNA has thymine (T)). In contrast, modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In other embodiments, the modification comprises one or more backbone alterations, such as a pyrimidine comprising a 2′-fluoro ribose structure, a C5-halogenated pyrimidine, a phosphorothioate group, or a 2′-O-methyl ribose structure. In a preferred embodiment, the modification comprises a 2′-fluoro ribose structure modification. In an even more preferred embodiment, the modification comprises a 2′-O-methyl ribose structure modification.

In some embodiments, the modifications may include nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an RNA molecule may have one or more nucleotides with a sugar modification at one position and also one or more nucleotides with a sugar modification at a different position.

Sugar modifications contemplated in the miRNA mimetic compounds include, but are not limited to, a substituent group selected from: OH; F; O-, S-, or N-alkyl; O-, S-, or N-alkenyl; O-, S-, of N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl.

In some embodiments, miRNA mimetic compounds have a sugar substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, or similar substituents. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked oligonucleotides and the 5′ position of 5′ terminal nucleotide.

In certain embodiments, the sugar modification is a 2′-O-alkyl (e.g., 2′-O-methyl, 2′-0-methoxyethyl), 2′-halo (e.g., 2′-fluoro, 2′-chloro, 2′-bromo), and 4′ thio modifications. For instance, in some embodiments, the first strand of the miR-25 mimetic compound comprises one or more 2′-fluoro nucleotides. In another embodiment, the first strand of the mimetic compounds has no modified nucleotides. In yet another embodiment, the second strand of miR-25 mimetic compound comprises one or more 2′-O-methyl modified nucleotides.

The first and the second strand of microRNA mimetic compounds of the invention can also include backbone modifications, such as one or more phosphorothioate, morpholino, or phosphonocarboxylate linkages (see, for example, U.S. Pat. Nos. 6,693,187 and 7,067,641, which are herein incorporated by reference in their entireties). For example, in some embodiments, the nucleotides comprising the 3′ overhang in the first strand and/or second strand are linked by phosphorothioate linkages. =The phosphorothioate bond substitutes a sulphur atom for a non-bridging oxygen in the phosphate backbone of an oligo. This modification renders the internucleotide linkage resistant to nuclease degradation. In some preferred embodiments, the first two nucleotides at the 5′ terminus of the second strand are linked by a phosphorothioate linkage.

In some embodiments, the microRNA mimetic compounds are conjugated to a carrier molecule such as a steroid (cholesterol), a vitamin, a fatty acid, a carbohydrate or glycoside, a peptide, or other small molecule ligand to facilitate in vivo delivery and stability. Preferably, the carrier molecule is attached to the second strand of the microRNA mimetic compound at its 3′ or 5′ end through a linker or a spacer group. In various embodiments, the carrier molecule is cholesterol, a cholesterol derivative, cholic acid or a cholic acid derivative. The use of carrier molecules is disclosed, for example, in U.S. Pat. No. 7,202,227, which is incorporated by reference herein in its entirety, is also envisioned. In certain embodiments, the carrier molecule is cholesterol and it is attached to the 3′ or 5′ end of the second strand through at least a six carbon linker. In some embodiments, the linker is a cleavable linker. In various embodiments, the linker comprises a substantially linear hydrocarbon moiety. The hydrocarbon moiety may comprise from about 3 to about 15 carbon atoms. In certain embodiments, the hydrocarbon linker/spacer comprises an optionally substituted C₂ to C₁₅ saturated or unsaturated hydrocarbon chain (e.g., alkylene or alkenylene). A variety of linker/spacer groups described in U.S. Pat. No. 9,012,225, which is incorporated by reference herein in its entirety, can be used in the present invention.

### 3. Pharmaceutical Compositions

The present disclosure also provides pharmaceutical compositions comprising a therapeutically effective amount of one or more miR-25 mimetic compounds described above and/or elsewhere herein, and a pharmaceutically acceptable carrier or excipient. In line with the invention, the first strand of the mimetic compound typically comprises a mature miR-25-3p sequence, and the second strand is substantially complementary to the first strand.

The invention also encompasses embodiments where additional therapeutic agents may be administered along with miR-25 mimetic compound. In one embodiment, the additional therapeutic agent is a second anti-fibrotic agent. The additional therapeutic agents may be administered concurrently but in separate formulations or sequentially. In other embodiments, additional therapeutic agents may be administered at different times prior to or after administration of miR-25 mimetic compounds. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, liposomes and exosomes, may be used as delivery vehicles for miR-25 mimetic compounds. In some embodiments, miR-25 mimics of the present invention may be formulated into liposome particles, which can then be aerosolized for inhaled delivery.

Commercially available fat emulsions that are suitable for delivering the nucleic acids of the invention to target tissues include Intralipid®, Liposyn®, Liposyn® II, Liposyn® III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533; and 6,747,014; and International PCT Patent Publication No. WO03/093449, which are herein incorporated by reference in their entireties.

In certain embodiments, liposomes used for delivery are amphoteric liposomes such SMARTICLES® (Marina Biotech, Inc.) which are described in detail in U.S. Patent Publication No. 2011/0076322. The surface charge on the SMARTICLES® is fully reversible which make them particularly suitable for the delivery of nucleic acids. SMARTICLES® can be delivered via injection, remain stable, and aggregate free and cross cell membranes to deliver the nucleic acids.

One will generally desire to employ appropriate salts and buffers to render delivery vehicles stable and allow for uptake by target cells. Aqueous compositions of the present invention comprise an effective amount of the delivery vehicle comprising the miR-25 mimic (e.g., liposomes or other complexes) dissolved or dispersed in a pharmaceutically acceptable carrier or aqueous medium. The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the polynucleotides of the compositions.

In one embodiment, pharmaceutical compositions of the invention are formulated for pulmonary, nasal, intranasal or ocular delivery and can be in the form of powders, aqueous solutions, aqueous aerosols, nasal drops, aerosols, and/or ocular drops. Solid formulations for nasal/intranasal administration may contain excipients such as lactose or dextran. Liquid formulations for nasal/intranasal administration may be aqueous or oily solutions for use in the form of aerosols, nasal drops or metered spray. Formulations for pulmonary/nasal/intranasal administration may also include surfactants such as, for example, glycocholic acid, cholic acid, taurocholic acid, ethocholic acid, deoxycholic acid, chenodeoxycholic acid, dehydrocholic acid, glycodeoxycholic acid, salts of these acids, and cyclodextrins.

In some embodiments, formulations for pulmonary/nasal/intranasal administration via inhalation include, but are not limited to a dry powder formulation, a liposomal formulation, a nano-suspension formulation, or a microsuspension formulation.

In some embodiments, pharmaceutical compositions for pulmonary/nasal/intranasal delivery are administered using an inhalation device. The term “inhalation device” refers to any device that is capable of administering a miR-25 mimic composition to the respiratory airways of the subject. Inhalation devices include devices such as metered dose inhalers (MDIs), dry powder inhalers (DPIs), jet nebulizers, ultrasonic wave nebulizers, heat vaporizers, soft mist inhalers, thermal aerosol inhalers, electrohydrodynamic-based solution misting inhaler. Inhalation devices also include high efficiency nebulizers. In some embodiments, a nebulizer is a jet nebulizer, an ultrasonic nebulizer, a pulsating membrane nebulizer, a nebulizer comprising a vibrating mesh or plate with multiple apertures, a nebulizer comprising a vibration generator and an aqueous chamber, or a nebulizer that uses controlled device features to assist inspiratory flow of the aerosolized aqueous solution to the lungs of the subject. Nebulizers, metered dose inhalers, and soft mist inhalers deliver pharmaceuticals by forming an aerosol which includes droplet sizes that can easily be inhaled.

In some embodiments, a composition administered with a high efficiency nebulizer comprises one or more miR-25 mimics and pharmaceutically acceptable excipients or carriers such as purified water, mannitol, surfactants, and salts such as sodium chloride and sodium EDTA, etc.

The active compositions of the present invention may include classic pharmaceutical preparations. Administration of these compositions according to the present invention may be via any common route so long as the target tissue is available via that route. This includes oral, nasal (e.g., inhalational), ocular, or buccal. Alternatively, administration may be by intravenous, intradermal, subcutaneous, intraocular or intramuscular injection, or by direct injection into pulmonary, cardiac, hepatic, pancreas, kidney, tumour, or skin tissue. Pharmaceutical compositions comprising miRNA mimics may also be administered by catheter systems or systems that isolate coronary circulation for delivering therapeutic agents to the heart. Various catheter systems for delivering therapeutic agents to the heart and coronary vasculature are known in the art.

Some non-limiting examples of catheter-based delivery methods or coronary isolation methods suitable for use in the present invention are disclosed in U.S. Pat. Nos. 6,416,510; 6,716,196; and 6,953,466, International Patent Publication Nos. WO2005/082440 and WO2006/089340, and U.S. Patent Publication Nos. 2007/0203445; 2006/0148742, and 2007/0060907, which are all herein incorporated by reference in their entireties. Such compositions would normally be administered as pharmaceutically acceptable compositions as described herein.

In other embodiments of the invention, compositions comprising miR-25 mimics as described herein may be formulated as a coating for a medical device, such as a stent, balloon, or catheter. Particularly useful in methods of treating cardiac fibrosis in a subject, the miR-25 mimics can be used to coat a metal stent to produce a drug-eluting stent. A drug-eluting stent is a scaffold that holds open narrowed or diseased arteries and releases a compound to prevent cellular proliferation and/or inflammation. The mimetic compounds may be applied to a metal stent imbedded in a thin polymer for release of the agonists or inhibitors over time. Methods for device-based delivery and methods of coating devices are well known in the art, as are drug-eluting stents and other implantable devices. See, e.g., U.S. Pat. Nos. 7,294,329; 7,273,493; 7,247,313; 7,236,821; 7,232,573; 7,156,869; 7,144,422; 7,105,018; 7,087,263; 7,083,642; 7,055,237; 7,041,127; 6,716,242; and 6,589,286, and International PCT Publication No. WO2004/004602, which are herein incorporated by reference in their entireties. Thus, the present invention includes a medical device, such as a balloon, catheter, or stent, coated with a miR-25 mimic.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients (e.g., as enumerated above).

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules, drug-eluting stents or other coated vascular devices, and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous, intradermal, intraocular, and intraperitoneal administration.

The pharmaceutical compositions of the invention can be formulated readily using pharmaceutically acceptable carriers well known in the art into dosages suitable for administration. Such carriers enable the compounds of the invention to be formulated in dosage forms such as tablets, pills, capsules, liquids, gels, syrups, slurries, suspensions and the like, for administration to the subject to be treated. For example, a pharmaceutical composition formulated for oral ingestion will contain a suitable carrier, for example, selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to elicit a beneficial response in the patient over time, such as a reduction in the symptoms associated with the condition. The quantity of the therapeutic/prophylactic agent(s) to be administered may depend on the subject to be treated inclusive of the age, sex, weight and general health condition thereof. In this regard, precise amounts of the therapeutic/prophylactic agent(s) for administration will depend on the judgement of the practitioner. In determining the effective amount of the agent to be administered in the treatment or prophylaxis of the condition, the physician may evaluate tissue levels of a polypeptide antigen, and progression of the disease or condition. In any event, those of skill in the art may readily determine suitable dosages of the therapeutic and/or prophylactic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilisers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy, but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dosage forms of the therapeutic agents of the invention may also include injecting or implanting controlled releasing devices designed specifically for this purpose or other forms of implants modified to act additionally in this fashion. Controlled release of an agent of the invention may be affected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be affected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulphuric, acetic, lactic, tartaric, malic, succinic, etc. Salts tend to be more soluble in aqueous or other protonic solvents that are the corresponding free base forms.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above.

The compositions of the present invention are suitably pharmaceutical compositions. The pharmaceutical compositions often comprise one or more “pharmaceutically acceptable carriers”. These include any carrier which does not itself induce the production of antibodies harmful to the individual receiving the composition. Suitable carriers typically are large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. A composition may also contain a diluent, such as water, saline, glycerol, etc. Additionally, an auxiliary substance, such as a wetting or emulsifying agent, pH buffering substance, and the like, may be present. A thorough discussion of pharmaceutically acceptable components is available in Gennaro (2000) Remington: The Science and Practice of Pharmacy. 20th ed, ISBN: 0683306472.

The compositions of the present disclosure generally may be formulated in a neutral or salt form. Pharmaceutically acceptable salts include, for example acid addition salts (formed with the free amino groups of the protein) derived from inorganic acids (e.g., hydrochloric or phosphoric acids), or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, topical solutions, drug-eluting devices or other coated vascular devices, and the like. In specific embodiments, the miR-25 mimetic compound is formulated for administration via intravenous injection, intramuscular injection, subcutaneous injection, intranasal spray/inhalation. iontophoresis, subconjunctival injection, sub-tendon injection, an intravitreal injection, intracamerally, or topically.

Collodial dispersion systems, such as macromolecule complexes, nanocapsules, microspheres, beads, and lipid-based systems including oil-in-water emulsions, micelles, mixed micelles, liposomes and exosomes, may be used as delivery vehicles for miR-25 mimetic compounds. In some embodiments, the miR-25 mimetic of the present disclosure may be formulated into liposome particles.

In some embodiments, the pharmaceutical composition of the disclosure comprises 2, 3, 4, 5, 6, 7, 8, 9, 10 or more miR-25 mimetics of the disclosure. In some embodiments, the pharmaceutical composition comprises one or more miR-25 mimetics of the disclosure and one or more other microRNAs or microRNA mimetics, including without limitation miR-25 mimetics other than those disclosed herein.

### 4. Dosage

The present invention is generally concerned with therapeutic and prophylactic compositions. The compositions will comprise an effective amount of the compositions defined herein, such that an amount of the miR-25 mimetic can be produced in vivo so that an therapeutic effect is generated in the individual to which it is administered. The exact amount necessary will vary depending on the subject being treated; the age and general condition of the subject to be treated; the capacity of the subject's immune system to synthesize antibodies; the degree of protection desired; the severity of the condition being treated; and its mode of administration, among other factors. An appropriate effective amount can be readily determined by one of skill in the art. Thus, an effective amount will fall in a relatively broad range that can be determined through routine trials.

Dosage amount and interval may be adjusted individually to provide plasma levels of the active compound(s) which are sufficient to maintain target antigen-reducing effects or effects that ameliorate the disease or condition. Usual patient dosages for systemic administration range from about from about 1 μg-500 μg, 0.5 mg-200 mg, and commonly from about 20 μg-500 μg.

In some embodiments, the pharmaceutical composition comprises a therapeutically effective amount of a miR-25 mimetic compound and a pharmaceutically acceptable carrier, wherein the pharmaceutically acceptable carrier is selected from water for injection (WFI), 0.9% (w/v) sodium chloride, 5 mM phosphate buffer, 10 mM phosphate buffer, 25 mM phosphate buffer, 50 mM phosphate buffer, 85 mM phosphate buffer, and 100 mM phosphate buffer.

In some embodiments, the pharmaceutical composition comprises 0.2 mg/mL to 200 mg/mL of the miR-25 mimetic. In some embodiments, the pharmaceutical composition comprises 0.2 mg/mL to 10 mg/mL, 10 mg/mL to 50 mg/mL, 50 mg/mL to 90 mg/mL, or 90 mg/mL to 120 mg/mL of the miR-25 mimetic. In some embodiments, the pharmaceutical composition comprises 0.2 mg/mL to 1 mg/mL, 1 mg/mL to 5 mg/mL, 5 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 90 mg/mL, or 90 mg/mL to 120 mg/mL of the miR-25 mimetic. In some embodiments, the pharmaceutical composition comprises at least about 0.1 mg/ml, at least about 0.2 mg/ml, at least about 0.3 mg/mL, at least about 0.5 mg/mL, at least about 1 mg/mL, at least about 2 mg/mL, at least about 3 mg/mL, at least about 4 mg/mL, at least about 5 mg/mL, at least about 7 mg/mL, at least about 10 mg/mL, at least about 20 mg/mL, at least about 30 mg/mL at least about 40 mg/mL, at least about 50 mg/mL, at least about 60 mg/mL, at least about 70 mg/mL, at least about 80 mg/mL, at least about 90 mg/mL or at least about 100 mg/mL of the miR-25 mimetic.

In some embodiments, the pharmaceutical composition comprises about 100 mg/mL, about 70 mg/mL, about 35 mg/mL, about 7 mg/mL, about 3.5 mg/mL, about 0.7 mg/mL, or about 0.35 mg/mL of the miR-25 mimetic. In some embodiments, the pharmaceutical composition comprises about 70 mg/mL and the miR-25 mimetic. In some embodiments, the pharmaceutical composition comprises about 35 mg/mL of the miR-25 mimetic.

In some embodiments, the pharmaceutical composition has a pH of 4 to 9. In some embodiments, the pharmaceutical composition has a pH of about 4, about 4.5, about 5, about 5.5, about 6, about 6.5, about 7, about 7.5, about 8, about 8.5, or about 9.

Alternatively, one may administer the agent in a local rather than systemic manner, for example, via injection of the compound directly into a tissue, often in a depot or sustained release formulation. Furthermore, one may administer the agent in a targeted drug delivery system, for example, in a liposome coated with tissue-specific antibody. The liposomes will be targeted to and taken up selectively by the tissue.

For any compound used in the method of the invention, the effective dose can be estimated initially from cell culture assays. For example, a dose can be formulated in animal models to achieve a circulating concentration range that includes the IC50 as determined in cell culture (e.g., the concentration of a test agent, which achieves a half-maximal reduction in target antigen). Such information can be used to more accurately determine useful doses in a mammal.

Toxicity and therapeutic efficacy of the compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in the subject. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the subject's condition. (See for example Fingi et al., 1975, in “The Pharmacological Basis of Therapeutics”, Ch. 1 p 1).

The compositions of the present invention can be suitably formulated for injection. The composition may be prepared in unit dosage form in ampules, or in multidose containers. The polynucleotides may be present in such forms as suspensions, solutions, or emulsions in oily or preferably aqueous vehicles. Alternatively, the polynucleotide salt may be in lyophilised form for reconstitution, at the time of delivery, with a suitable vehicle, such as sterile pyrogen-free water. Both liquid as well as lyophilised forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Non-ionic materials, such as sugars, are preferred for adjusting tonicity, and sucrose is particularly preferred. Any of these forms may further comprise suitable formulatory agents, such as starch or sugar, glycerol or saline. The compositions per unit dosage, whether liquid or solid, may contain from 0.1% to 99% of polynucleotide material.

The unit dosage ampules or multidose containers, in which the polynucleotides are packaged prior to use, may comprise a hermetically sealed container enclosing an amount of polynucleotide or solution containing a polynucleotide suitable for a pharmaceutically effective dose thereof, or multiples of an effective dose. The polynucleotide is packaged as a sterile formulation, and the hermetically sealed container is designed to preserve sterility of the formulation until use.

The dosage to be administered depends to a large extent on the condition and size of the subject being treated as well as the frequency of treatment and the route of administration. Regimens for continuing therapy, including dose and frequency may be guided by the initial response and clinical judgment. The parenteral route of injection into the interstitial space of tissues is preferred, although other parenteral routes, such as inhalation of an aerosol formulation, may be required in specific administration, as for example to the mucous membranes of the nose, throat, bronchial tissue or lungs.

### 5. Methods of Treatment

In various embodiments, the present disclosure provides methods of treating, ameliorating, or preventing fibrotic conditions in a subject in need thereof comprising administering to the subject a therapeutically effective amount of miR-25 mimetic described herein.

Fibrotic conditions that may be treated using a miR-25 mimetic of the disclosure include, but are not limited to liver fibrosis, kidney fibrosis, lung fibrosis, cardiac fibrosis, skin fibrosis, age-related fibrosis, spleen fibrosis, scleroderma, and/or post-transplant fibrosis.

In certain embodiments, the fibrosis is liver fibrosis and is present in a subject having a disease selected from chronic liver injury, hepatitis infection (such as hepatitis B infection and/or hepatitis C infection), non-alcoholic steatohepatitis, alcoholic liver disease, liver damage following exposure to environmental toxin and/or natural product, and cirrhosis.

In certain embodiments, the pulmonary fibrosis is idiopathic pulmonary fibrosis, or the subject has chronic obstructive pulmonary disease.

In some embodiments, the fibrosis is kidney fibrosis and is present in a subject having a disease or condition selected from glomerulosclerosis, tubulointerstitial fibrosis, IgA nephropathy, intestinal fibrosis/tubular atrophy, chronic kidney damage, chronic kidney disease, glomerular disease, glomerulonephritis, diabetes mellitus, idiopathy focal segmental glomerulosclerosis, membranous nephropathy, collapsing glomerulopathy, chronic recurrent kidney infection, chronic kidney disease following acute kidney injury (AKI), kidney damage following exposure to environmental toxin and/or natural product, and end stage renal disease. In certain embodiments, the kidney fibrosis results from acute or repetitive trauma to the kidney.

In certain embodiments, the disease is an inflammatory disease.

In some embodiments, administration of a miR-25 mimetic of the present disclosure reduces the expression or activity of one or more extracellular matrix genes in cells of the subject. In another embodiment, administration of a miR-25 mimetic of the present disclosure reduces the expression or activity of one or more collagen synthesis genes in cells of the subject. Cells of the subject where the expression or activity of various genes is regulated by miR-25 mimetic of the disclosure in fibroblasts, keratocytes, epidermal, epithelial, endothelial cells and hepatic stellate cells. In some embodiments, administration of a miR-25 mimetic reduces the expression of COL1A1, COL1A2, COL3A1, COL4A3, COL5A2, COL11A1, FN1, MMP2, CTGF, TGFB2, and/or TGFB3. In some embodiments, administration of a miR-25 mimetic downregulated inflammatory responses associated with fibrosis (e.g., MCP1). In some embodiments, administration of a miR-25 mimetic reduces infiltration of immune effector cells such as neutrophils, lymphocytes, monocytes and macrophages in fibrotic tissues or organs. In some embodiments, administration of a miR-25 mimetic reduces or inhibits epithelial-to-mesenchymal transition. In some embodiments, administration of miR-25 mimetic reduces or inhibits myofibroblast differentiation.

In certain embodiments, the present disclosure provides methods of regulating an extracellular matrix gene in a hepatic stellate cell comprising contacting the cells with a miR-25 mimetic of the present disclosure. In some embodiments, the disclosure provides methods of regulating a collagen synthesis gene in a hepatic stellate cell comprising contacting the hepatic stellate cells with a miR-25 mimetic of the present disclosure. Upon treatment or contact, the miR-25 mimetic reduces the expression or activity of the extracellular matrix gene or the collagen synthesis gene.

In some embodiments, the disclosure provides methods of treating, preventing, decreasing, or diminishing fibrosis secondary to treatment of the organ with an antibody, a small-molecule drug, a biologic drug, an aptamer, or a virus (e.g., a viral vector).

The disclosure also provides methods for assessing the efficacy of a fibrosis treatment with a miR-25 agonist (e.g., drug or miR-25 mimetic). For instance, in some embodiments, the method for assessing the treatment efficacy comprises determining a level of expression of one or more genes in hepatic stellate cells of a subject prior to the treatment with a miR-25 mimetic, wherein the one or more genes are selected from a set of genes modulated by miR-25, determining the level of expression of the same one or more genes in cells/fibrotic tissue of the subject after treatment with a miR-25 mimetic; and determining the treatment to be effective, less effective, or not effective based on the expression levels prior to and after the treatment. In another embodiment, at least 1-fold, 1.5-fold, 2-fold, 2.5-fold, 3-fold, 3.5-fold, or 4-fold difference in the expression of the genes prior to and after treatment indicates the treatment to be effective.

### 6. Kits

The present invention also provides kits comprising an miR-25 mimetic as broadly described above and elsewhere herein. Such kits may additionally comprise alternative immunogenic agents for concurrent use with the immunostimulatory compositions of the invention.

In some embodiments, in addition to the immunostimulatory compositions of the present invention the kits may include suitable components for performing the prime-boost regimens described above. For example, the kit may include separately housed priming and boosting doses of the at least one polypeptide antigens.

The kits may comprise additional components to assist in performing the methods of the present invention such as, for example, administration device(s), buffer(s), and/or diluent(s). The kits may also include containers for housing the various components and instructions for using the kit components in the methods of the present invention.

In order that the invention may be readily understood and put into practical effect, particular preferred embodiments will now be described by way of the following non-limiting examples.

### EXAMPLES

### Example 1

### Novel MIR-25 Mimetics Efficiently Downregulates Target Genes FKBP14 and ADAM-17

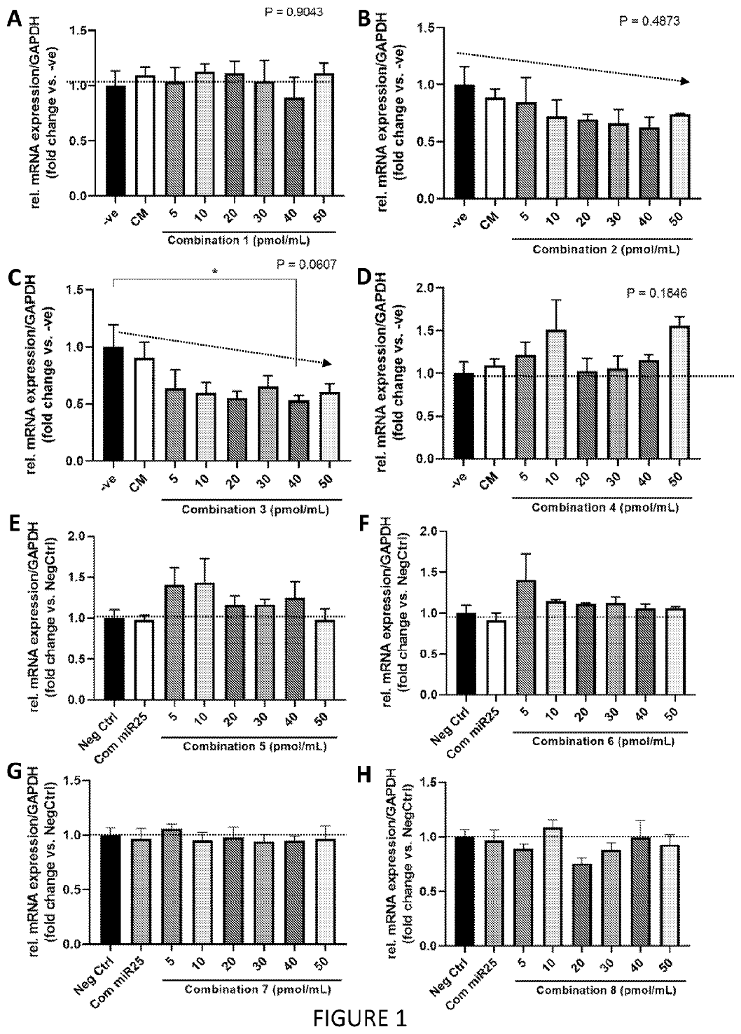

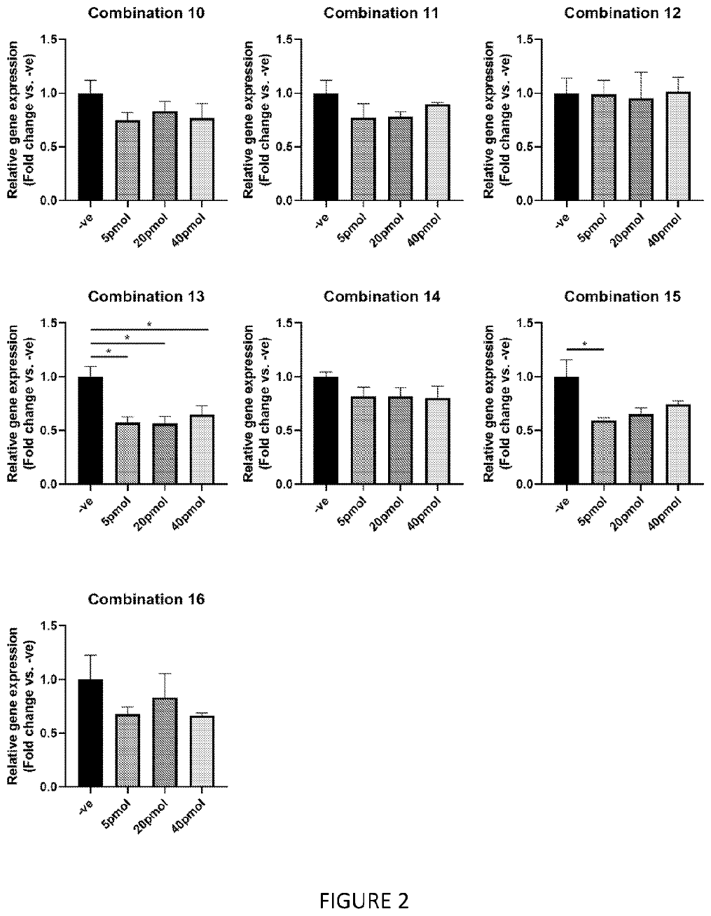

The inventors previously demonstrated that the Notch-signalling regulators FKBP14 and ADAM-17 are direct targets of miR-25 and are downregulated by miR-25 overexpression in HSCs using commercial miR-25 mimetics (Genz, B et al. Scientific Reports, 2019). Seeking to improve efficiency of target gene downregulation, the inventors designed 15 proprietary mimetics (Table 4; Combination 1-16, C1-16) based on the mature miR-25 sequence (SEQ ID NO: 1). Out of the 15 mimetics, C2, C3, C13 and C15 showed concentration-dependent downregulation of FKBP14 using 5 to 40 pmol/mL mimetic, with C3 presenting the highest target gene downregulation of up to 50% (p<0.05 at 40 pmol/ml; FIG. 1 and FIG. 2). C3, C13 and C15 were therefore used in further experiments at 5 pmol/mL, 20 pmol/mL and 40 pmol/mL to evaluate effects of low and high miR-25 concentrations on target genes.

TABLE 4

| | SEQ | |
|---|---|---|
| | ID | |
| C | NO | Sequence (5′ to 3′) |
| 1 | 2 | 5′-rC*rA.rU.rU.rG.rC.rA.rC.rU.rU.rG.rU.rC.rU.rC.rG.rG.rU.rC.rU*rG*rA*rU*rU-3′ |
| | 3 | 5′-rU*rC.rA.rG.rA.rC.rC.rG.rA.rG.rA.rC.rA.rA.rG.rU.rG.rC*rA*rA*rU*rG*rU*rU-3′ |
| 2 | 4 | 5′-rC*rA.rU.rU.rG.rC.rA.rC.rU.rU.rG.rU.rC.rU.rC.rG.rG.mU.mC.mU*mG*mA*mU*mU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 3 | 2 | 5′-rC*rA.rU.rU.rG.rC.rA.rC.rU.rU.rG.rU.rC.rU.rC.rG.rG.rU.rC.rU*rG*rA*rU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 4 | 3 | 5′-rU*rC.rA.rG.rA.rC.rC.rG.rA.rG.rA.rC.rA.rA.rG.rU.rG.rC*rA*rA*rU*rG*rU*rU-3′ |
| | 4 | 5′-rC*rA.rU.rU.rG.rC.rA.rC.rU.rU.rG.rU.rC.rU.rC.rG.rG.mU.mC.mU*mG*mA*mU*mU-3′ |
| 5 | 6 | 5′-rC*mA.rU.rU.rG.rC.rA.rC.rU.rU.rG.rU.rC.rU.rC.rG.rG.rU.rC.rU*rG*rA*mU*LT-3′ |
| | 7 | 5′-mU*rC.mA.rG.mA.rC.mC.rG.mA.rG.mA.rC.mA.rA.mG.rU.mG.rC.mA*rA*mU*rG*mU*LT-3′ |
| 6 | 8 | 5′-rC*mA.rU.rU.rG.rC.rA.rC.rU.rU.rG.rU.rC.rU.rC.rG.rG.rU.rC.rU*rG*rA*rU*LT-3′ |
| | 7 | 5′-mU*rC.mA.rG.mA.rC.mC.rG.mA.rG.mA.rC.mA.rA.mG.rU.mG.rC.mA*rA*mU*rG*mU*LT-3′ |
| 7 | 9 | 5′-rC*rA.rU.rU.rG.rC.rA.rC.rU.rU.rG.rU.rC.rU.rC.rG.rG.rU.rC.rU*rG*rA*mU*LT-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 8 | 10 | r5′-C*rA.rU.rU.rG.rC.rA.rC.rU.rU.rG.rU.rC.rU.rC.rG.rG.rU.rC.rU*rG*rA*rU*LT-3′ |
| | 5 | 5′-mU*rC.rA.rG.rA.rC.rC.rG.rA.rG.rA.rC.rA.rA.rG.rU.rG.mC*mA+mA*mU+mG+mU+mU |
| 10 | 11 | 5′-rC*mA.rU.rU.rG.rC.rA.rC.rU.rU.rG.rU.rC.rU.rC.rG.rG.rU.rC.rU*rG*rA*rU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 11 | 12 | 5′-rC*rA.rU.rU.rG.rC.rA.rC.rU.rU.rG.rU.rC.rU.rC.rG.rG.rU.rC.rU*rG*rA*mU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 12 | 13 | 5′-rC*mA.rU.rU.rG.rC.rA.rC.rU.rU.rG.rU.rC.rU.rC.rG.rG.rU.rC.rU*rG*rA*mU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 13 | 14 | 5′-rC*rA.rU.rU.rG.rC.rA.rC.rU.rU.rG.rU.rC.rU.rC.rG.rG.mU.rC.mU.rG*rA*rU*rU-3′ |
| | 5 | 5′-mU*.rC.rA.rGrArC.rCr.GrA.rG.rA.rC.rA.rA.rG.rU.rG.mC*.mA*.mA*.mU*.mG*.mU*. |
| | | mU-3′ |
| 14 | 15 | 5′-rC*rA.fU.rU.rG.fC.rA.fC.rU.rU.rG.rU.rC.rU.rC.rG.rG.rU.rC.rU*rG*rA*rU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 15 | 16 | 5′-rC*rA.fU.rU.rG.fC.rA.fC.rU.rU.rG.rU.rC.rU.rC.rG.rG.mU.rC.mU.rG*rA*rU.rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |
| 16 | 17 | 5′-rC*rA.fU.rU.rG.fC.rA.fC.rU.rU.rG.rU.rC.rU.rC.rG.rG.fU.rC.fU*rG*rA*rU*rU-3′ |
| | 5 | 5′-mU*rC.rA.rG.rA.rC.rC.rG.rA.rG.rA.rC.rA.rA.rG.rU.rG.mC*mA*mA*mU*mG*mU*mU-3′ |

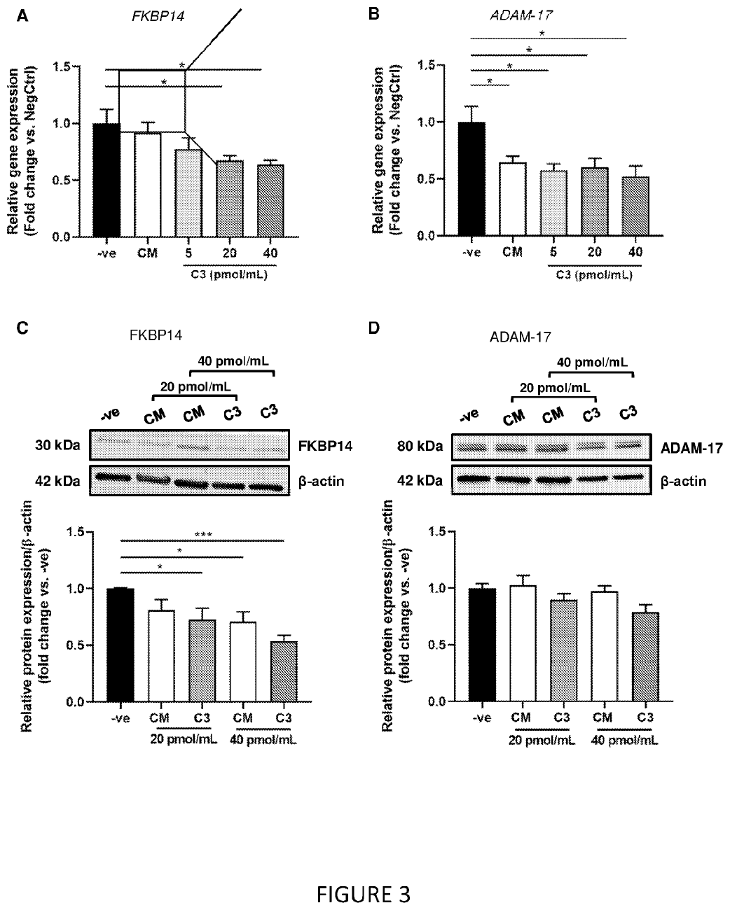

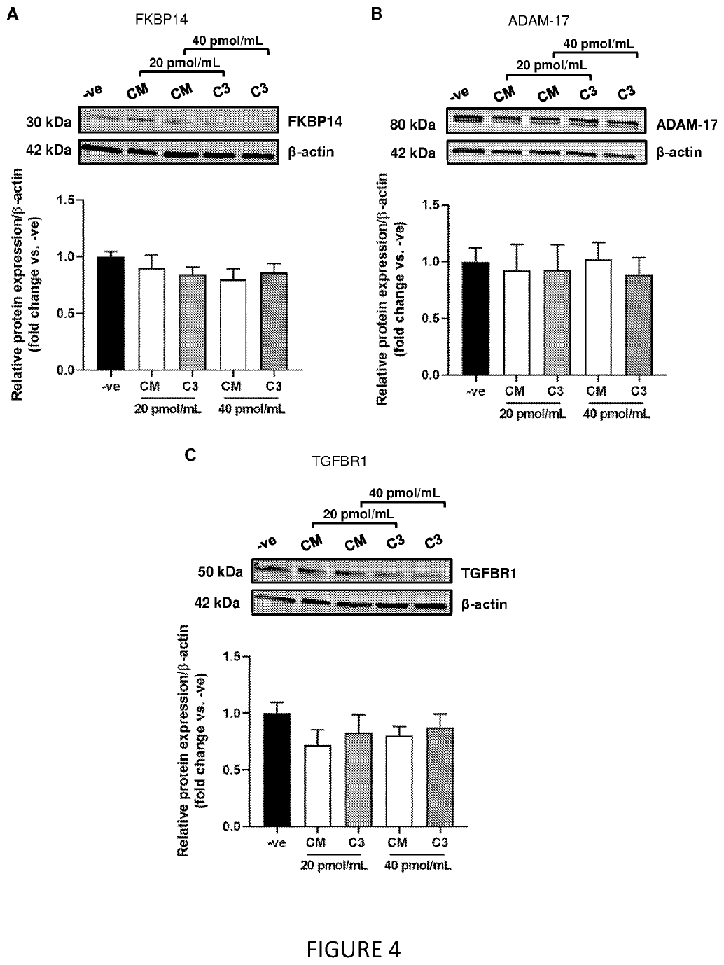

mRNA expression of both FKBP14 and ADAM-17 was downregulated using C3, compared to the negative control and the commercial mimetic. Downregulation of FKBP14 was statistically significant compared to the negative control using 20 and 40 pmol/mL (p<0.05; FIG. 3A) of C3, which was a ˜30% further decrease in mRNA compared to the commercial mimetic. Downregulation of ADAM-17 was statistically significant using the commercial mimetic, 5, 20 and 40 pmol/mL of C3 (p<0.05; FIG. 3B). Initial analysis of protein expression 48 h after transfection with miR-25 mimetics showed no significant decreases for neither FKBP14 nor ADAM-17 (FIG. 4). Protein expression of FKBP14 was significantly downregulated after 72 h using 40 pmol/mL of commercial miR-25 (p<0.05), and both 20 pmol/mL (p<0.05) and 40 pmol/mL (p<0.001) of C3 (FIG. 3B). ADAM-17 protein expression was downregulated after 72 h using C3, compared to both the negative control and commercial mimetic; however, the results did not reach statistical significance (FIG. 3B).

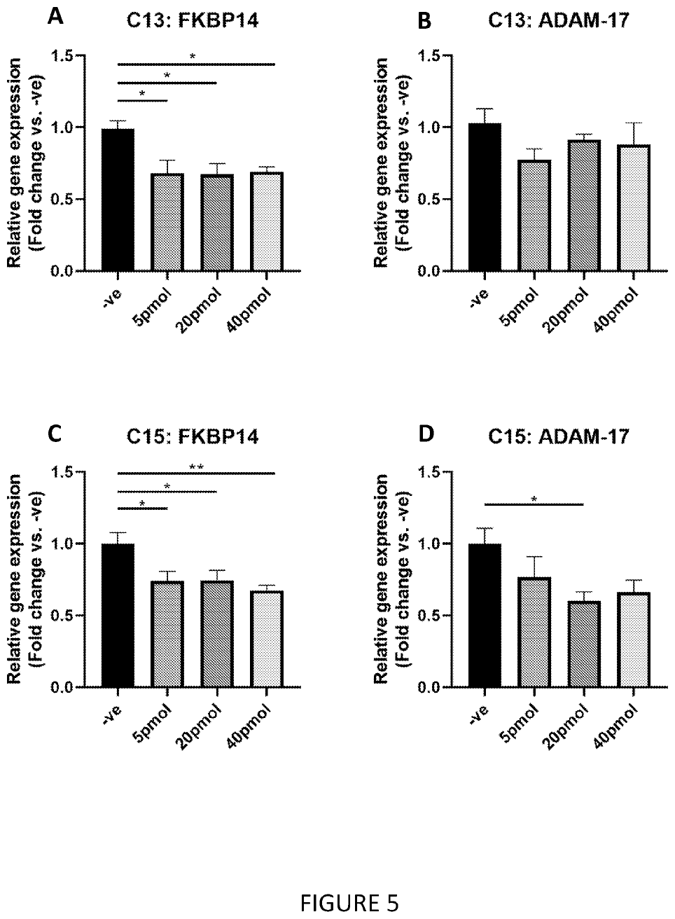

For C13 and C15, mRNA expression of both FKBP14 and ADAM-17 was downregulated when compared to the negative control. Downregulation of FKBP14 was statistically significant for both C13 and C15 when compared to the negative control using 5, 20 and 40 pmol/mL (p<0.05; FIGS. 5A & 5C). Downregulation of ADAM-17 was statistically significant for 20 pmol/mL of C15 only, when compared to the negative control (p<0.05; FIG. 5D). ADAM-17 protein expression was downregulated after 72 h using C13, compared to the negative control; however, the results did not reach statistical significance (FIG. 5B).

### Example 2

### Novel MIR-25 Mimetics Inhibits Expression of Collagens

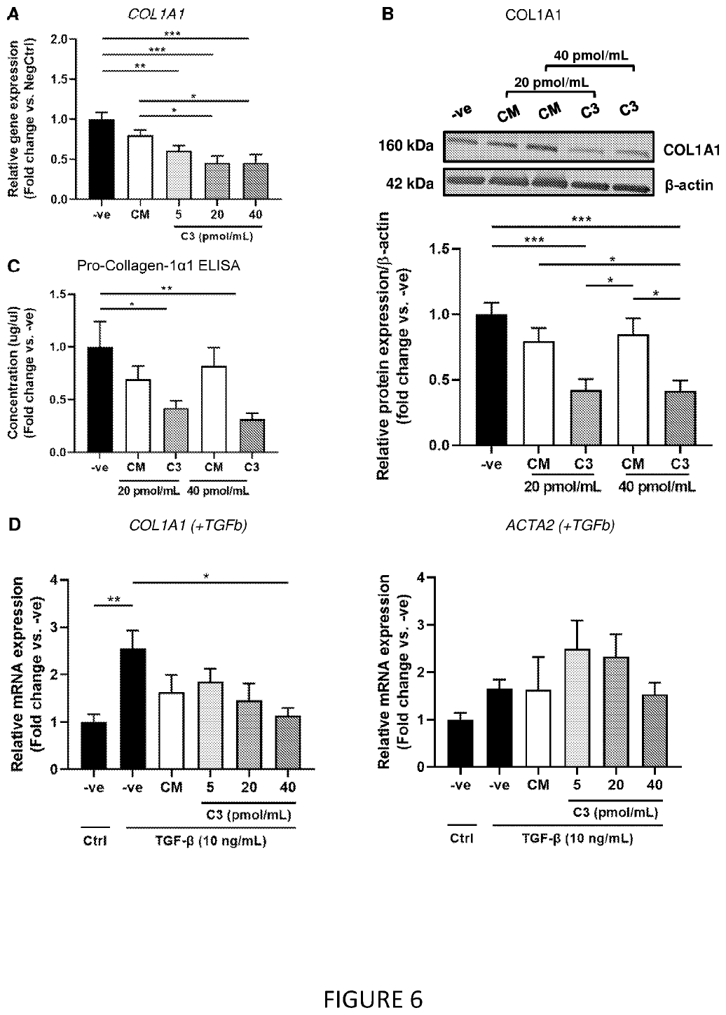

In extra-hepatic cell types, miR-25 has been shown to downregulate fibrillar collagen expression. Of great interest in this present application, C3 significantly downregulated mRNA expression of fibrillar collagens type I and Ill. COL1A1 mRNA was significantly downregulated compared to negative control using 5 pmol/mL (p<0.01), 20 pmol/mL and 40 pmol/mL (p<0.001) of C3. Compared with the commercial mimetic, downregulation of COL1A1 mRNA was significantly improved by ˜35% using C3 at 20 pmol/mL and 40 pmol/mL (p<0.05; FIG. 6A). Accordingly, COL1A1 protein expression was significantly downregulated after 72 h using C3 at 20 pmol/mL and 40 pmol/mL compared to the negative control (p<0.001; FIG. 6B). Compared with the commercial miR-25 at 40 pmol/mL, C3 further downregulated COL1A1 protein expression by ˜43% using both 20 pmol/mL and 40 pmol/mL (p<0.05; FIG. 6B). Similarly, Pro-Collagen1α1 expression in LX-2 cell culture supernatant was significantly decreased after 72 h using C3 at 20 pmol/mL (p<0.05) and 40 pmol/mL (p<0.01) compared to the negative control (FIG. 6C).

TGF-β stimulation significantly upregulated COL1A1 mRNA expression in LX-2 cells transfected with the negative control mimetic (p<0.01; FIG. 6D; left panel). This TGF-β-dependent COL1A1 induction was inhibited in cells transfected with C3, reaching statistical significance at 40 pmol/mL (p<0.05; FIG. 6D; left panel). Compared to the commercial miR-25 mimetic, C3 reduced TGF-β-induced COL1A1 expression by a further ˜20%, although did not reach statistical significance (FIG. 6D; left panel). miR-25 overexpression had no evident effect on TGF-β-induced ACTA2 mRNA expression (FIG. 6D; right panel).

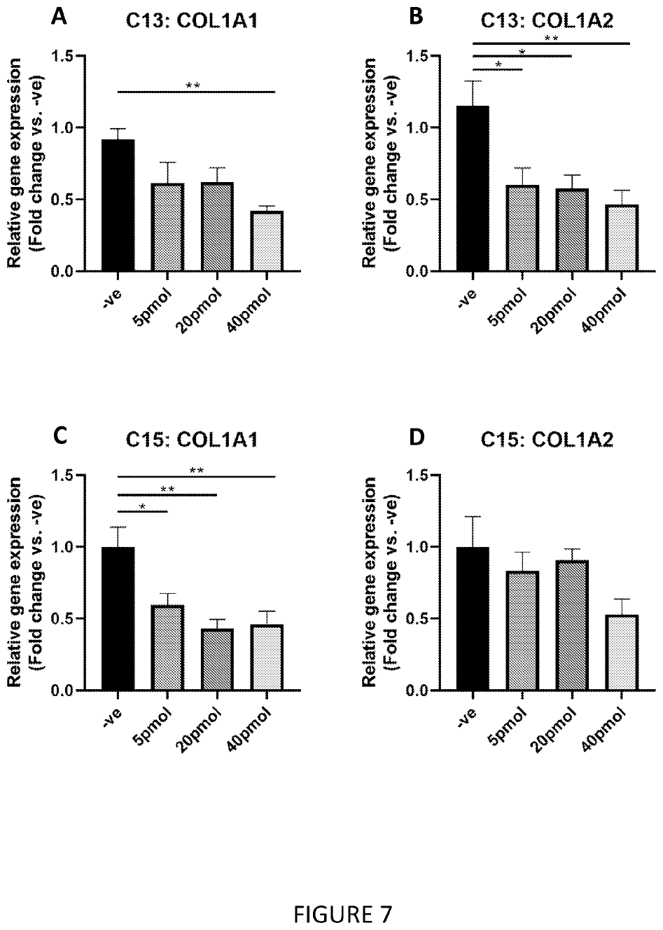

For C13 and C15, mRNA expression of both COL1A1 and COL1A2 was downregulated when compared to the negative control. Downregulation of COL1 A1 was statistically significant for both C13 using 40 pmol/mL and C15 using 5, 20 and 40 pmol/mL, when compared to the negative control (p<0.05; FIGS. 7A & 7C). Downregulation of COL1 A2 was statistically significant for 5, 20 and 40 pmol/mL of C13 only, when compared to the negative control (p<0.05; FIG. 5B). COL1A2 protein expression was downregulated after 72 h using C15, compared to the negative control; however, the results did not reach statistical significance (FIG. 7D).

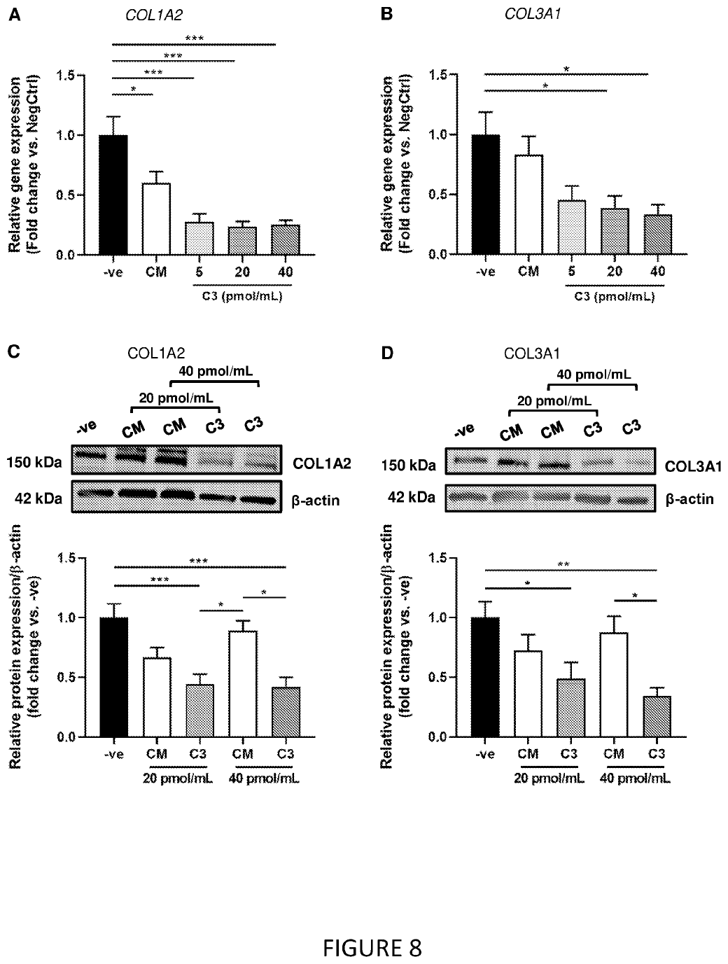

COL1A2 mRNA was also significantly downregulated using all three concentrations of C3 (p<0.001), compared to the negative control (FIG. 8A). Similarly, COL1A2 protein expression was significantly downregulated after 72 h using C3 at 20 pmol/mL and 40 pmol/mL, compared to the negative control (p<0.01; FIG. 8B). COL3A1 mRNA was also significantly downregulated compared to the negative control using 20 pmol/mL and 40 pmol/mL of C3 (p<0.05; FIG. 8C). Further to this, COL3A1 protein expression was markedly downregulated after 72 h using C3 at 20 pmol/mL compared to the negative control (p<0.05) and at 40 pmol/mL, compared to both the negative control (p<0.001) and 40 pmol/mL of the commercial mimetic (p<0.05; FIG. 8D).

### Example 3

### C3 has Minor Effects on Non-Fibrillar Collagen Expression

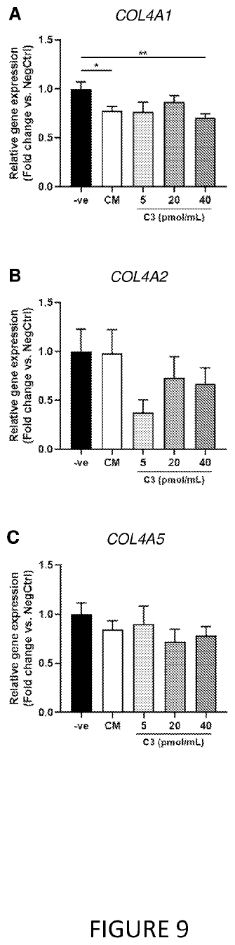

COL4A1 mRNA expression was significantly downregulated compared to the negative control using the commercial mimetic 20 pmol/mL (p<0.05) and 40 pmol/mL (p<0.01) of C3 (FIG. 9A). COL4A2 mRNA expression was downregulated by ˜60%, 25% and 30% using 5, 20 and 40 pmol/mL of C3, respectively, compared to both the negative control and commercial mimetic, although this did not reach statistical significance (FIG. 9B). miR-25 overexpression did not significantly downregulate COL4A5 mRNA (FIG. 9).

### Example 4

### Novel MIR-25 Mimetic Selectively Downregulates TGF-BR1

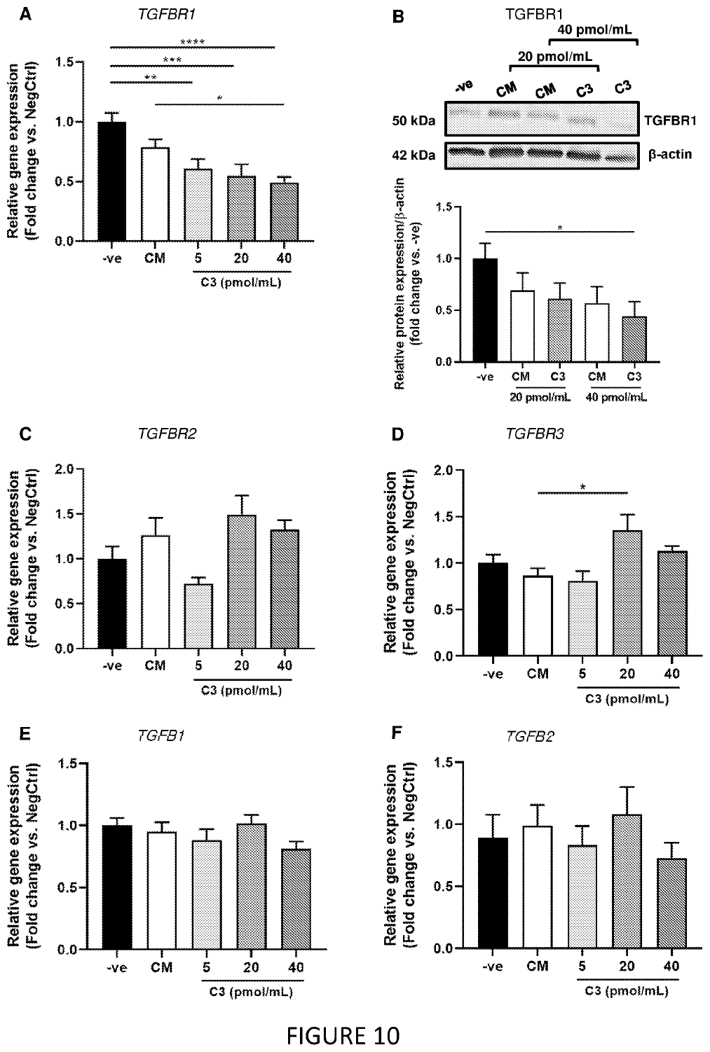

The inventors further investigated the effect of miR-25 overexpression on TGF-β signalling genes. TGF-βR1 mRNA was significantly downregulated in a concentration-dependent manner using C3 at 5 pmol/mL (p<0.01), 20 pmol/mL (p<0.001) and 40 pmol/mL (p<0.0001), compared to the negative control (FIG. 10A). TGF-βR1 mRNA was also significantly downregulated using 40 pmol/mL of C3 compared to the commercial mimetic (20 pmol/ml; p<0.05; FIG. 10A). This same effect was reflected in TGF-βR1 protein expression after 72 h, with 40 pmol/mL of C3 downregulating expression by ˜55% compared to the negative control (p<0.05; FIG. 10). TGF-βR2, TGF-βR3, TGF-β1 and TGF-β2 mRNA expression were not affected by miR-25 overexpression (FIG. 10C-F).

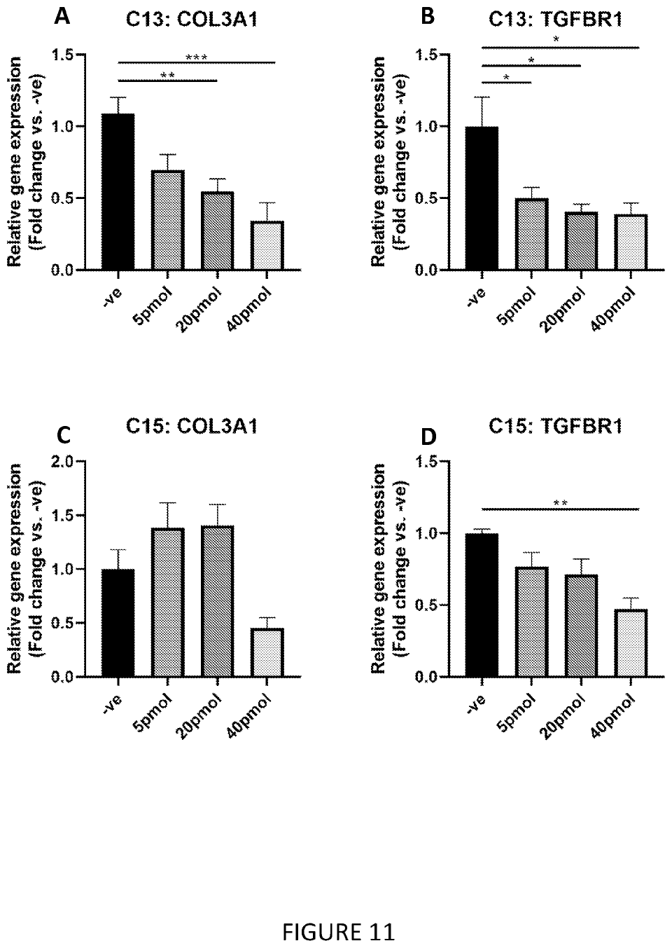

COL3A1 mRNA was significantly downregulated compared to the negative control using 20 pmol/mL and 40 pmol/mL of C13 (p<0.05; FIG. 11A). The inventors further investigated the effect of miR-25 overexpression on TGF-β signalling genes. TGF-βR1 mRNA was significantly downregulated in a concentration-dependent manner using C13 and C15 at 5 pmol/mL (p<0.01), 20 pmol/mL (p<0.001) and 40 pmol/mL (p<0.0001), compared to the negative control (FIGS. 11B & D).

### Example 5

### C3 Specifically Downregulates Collagen Modulating Factors Via Tgfb Signalling Regulation

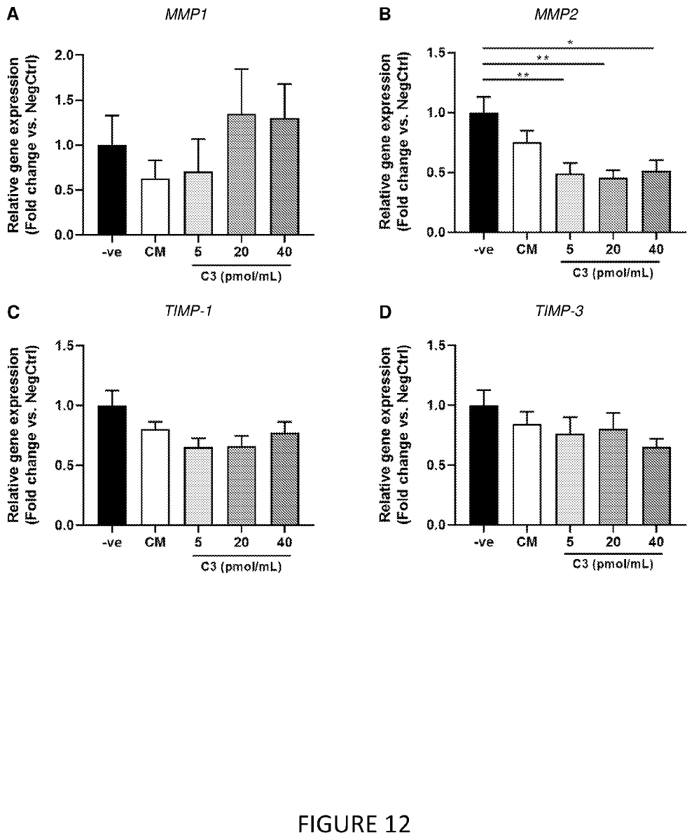

To broaden the knowledge of miR-25's function in HSCs, the inventors analysed its effect on expression of collagen modulating factors that are increased during HSC activation and fibrosis. miR-25 had no effect on MMP1 expression (FIG. 12A). MMP2 mRNA was significantly downregulated compared to the negative control using C3 at 5 and 20 pmol/mL (p<0.01) and 40 pmol/mL (p<0.05; FIG. 12B). TIMP-1 and TIMP-3 mRNA expression were downregulated with miR-25 overexpression, but not to a statistically significant effect (FIG. 12C-D).

### Example 6

### C3 Decreases Cell Proliferation, but does not Affect Cell Migration Nor Cell Contractility

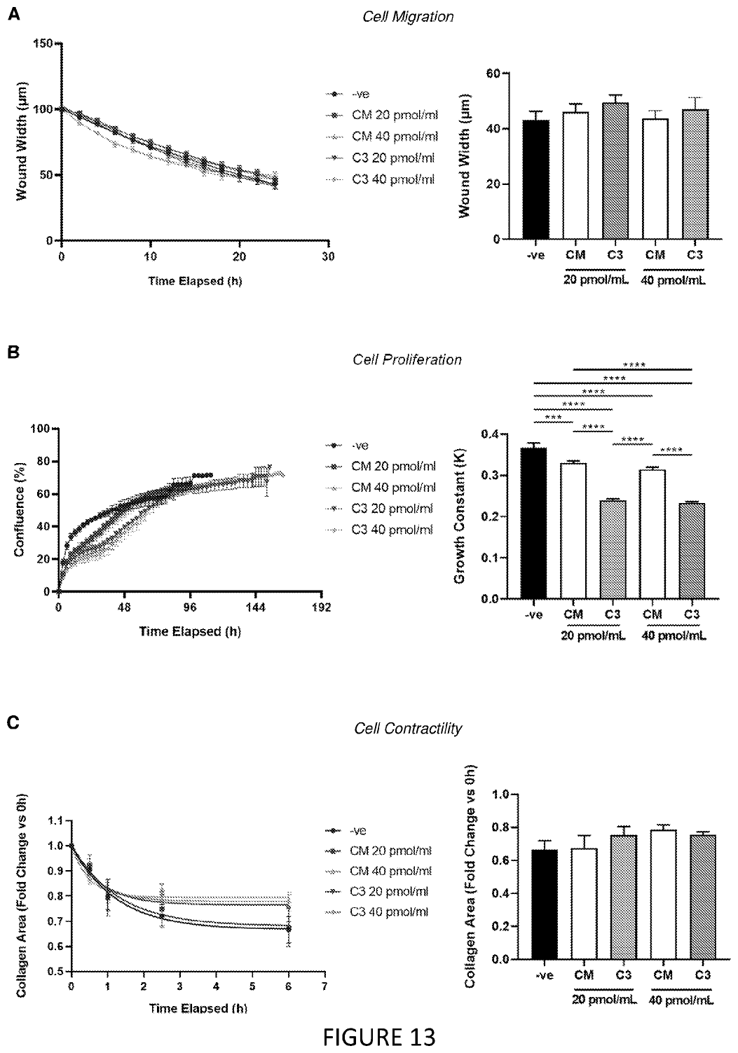

Cell proliferation, migration and contractility are increased during HSC activation. The inventors examined if miR-25 overexpression affects this phenotype. Wound width closure rate (μm) was not significantly upregulated using C3 compared to the commercial mimetic and negative control (FIG. 13A). Cell proliferation rate was significantly decreased using C3 at both 20 pmol/mL and 40 pmol/mL compared to the negative control (p<0.0001) and compared to both concentrations of the commercial miR-25 mimetic (p<0.0001; FIG. 13B). Commercial miR-25 also significantly decreased cell proliferation compared to the negative control, but to a lesser extent compared to C3 (p<0.001 at 20 pmol/mL, p<0.001 at 40 pmol/ml; FIG. 13B). Cell contractility was not significantly affected using C3 compared to the commercial mimetic and negative control (FIG. 13C).

### Materials & Methods

6.1 Proprietary miR-25-3p Mimetic Synthesis and Annealing.

Chemically modified, single strand RNA sequences were synthesised based on the mature miR-25-3p sequence. RNA sequences were annealed in 1 mL of 1×annealing buffer solution (10 mM UltraPure Tris, 50 mM NaCl, 1 mM EDTA in Ultrapure water) by heating up to 85° C. before cooling down to room temperature. Different combinations of sequences were annealed to create proprietary miRNA mimetics (Table 4). Proprietary mimetics include RNA nucleotides (rA/rG/rC/rU) containing conventional phosphate and phosphorothioate (*) linkages as well as 2′-O-Methyl-RNA nucleotides (mA/mG/mC/mU) or 2′-Fluoro-RNA nucleotides (fA/fG/fC/fU) (Table 3).

6.2 Cell Culture and miRNA Mimetic Transfection

All experiments were conducted in vitro in cultured LX-2 cells, an immortalised human HSC line (provided by Prof. Scott L. Friedman, Mount Sinai School of Medicine, NY, USA). LX-2 cells were cultured in Dulbecco's Modified Eagle Medium (DMEM, high glucose; Sigma Aldrich, St. Louis, MO, USA), containing 2% fetal calf serum (FCS), 1% glutamine, and 1% penicillin/streptomycin at 37° C. and 5% C02. Cells were cultured in 6-well tissue culture plates (2×105 cells/well for RNA isolation) or 10 cm cell culture dishes (5×10⁵ cells/dish for protein extraction) until 80% confluency. Cells were transiently transfected 24-hours after cell seeding with C3, commercial miR-25 mimetic or negative control (MISSION microRNA, Sigma) at 5, 20 or 40 pmol/mL, using Lipofectamine LTX (Life Technologies, Carlsbad, CA, USA) according to the manufacturer's protocol. The negative control used was a non-specific miRNA mimetic from a Caenorhabditis elegans sequence (MISSION microRNA mimetics negative control 2; Sigma). Cells were harvested 48 h after transfection in 350 μl RLT buffer (Qiagen, Hilden, Germany) for RNA isolation, or 100 μl RIPA buffer (2% NP-40, 0.05% Na-deoxycholate, 0.1% sodium dodecyl sulfate (SDS), 1×protease and phosphatase inhibitor in PBS) for protein isolation and stored at −20° C. until further use.

### 6.3 RNA Isolation and αRT-PCR Analysis

RNA was isolated from transfected LX-2 cells using the RNeasy Mini Kit (Qiagen) following the manufacturer's protocol. RNA concentration was quantified using the NanoDrop™ spectrophotometer (ThermoFisher Scientific, Waltham, MA, USA). 100 ng of isolated RNA was transcribed to complementary DNA (cDNA) using SensiFAST™ cDNA synthesis kit (Bioline, Luckenwalde, Germany) under the following conditions: priming for 10 min at 25° C., reverse transcription for 15 min at 420C, and inactivation for 5 min at 85° C. cDNA was diluted 1:2 in nuclease-free water and stored at −20° C. Quantitative real-time polymerase chain reaction (qRT-PCR) was conducted using custom primer sequences listed in Table 5. qPCR was performed with Platinum SYBR Green qPCR SuperMix-UDG (Life Technologies) using a standard protocol (UDG incubation for 2 min at 50° C., hold for 2 min at 95° C.; 40 cycles of: 95° C. for 15 sec, 600C for 30 sec) in the CFX384 Touch™ Thermal Cycler (BioRad). Target gene mRNA expression was normalised to the relative expression of the housekeeping gene Glyceraldehyde 3-phosphate dehydrogenase (GAPDH) and analysed using the 2 ACT method. Relative expression values for each treatment were normalised to the average value for negative control samples to calculate fold change.

TABLE 5
PRIMER SEQUENCES USED FOR QRT-PCR
Custom Primers

| Human Gene | Primer | Sequence (5′ to 3′) |
|---|---|---|
| ACTA2 | Forward | CTATCAGGGGGCACCACTATGTA |
| | Reverse | GCTCCGGAGGGGCAATGA |
| ADAM-17 | Forward | CCATGAAGTGTTCCGATAGAT |
| | Reverse | ACCTGAAGAGCTTGTTCATCG |
| COL1A1 | Forward | AAGCCGAATTCCTGGTCTGG |
| | Reverse | CGATGGCTGCACGAGTCACA |
| COL1A2 | Forward | TGCTAAAGGAGAAAGAGGAGCCA |
| | Reverse | CCATCACCACGACTTCCAGC |
| COL3A1 | Forward | GCGCCTCCTAATGGTCAAGG |
| | Reverse | TTCTGAGGACCAGTAGGGCA |
| COL4A1 | Forward | ACCATAGAGAGGAGCGAGATGTTC |
| | Reverse | CTCATACAGACTTGGCAGCGG |
| COL4A2 | Forward | GAGCCTGGATTGGTCGGTTTC |
| | Reverse | AGTCCTCTGTTGCCTTGCTGT |
| COL4A5 | Forward | CCCGTGGTTTGGATGGTCCC |
| | Reverse | TTGTCTGGCTGTGGCGTGTAA |
| FKBP14 | Forward | ACGGCTCCTTATTTCACTCCAC |
| | Reverse | ACACATTCCTTTCAAGCCCT |
| GAPDH | Forward | GTCCACCACCCTGTTGCTGTAG |
| | Reverse | GACACCCACTCCTCCACCTTTGA |
| HES1 | Forward | CTGAGCACAGACCCAAGTGT |
| | Reverse | GAGTGCGCACCTCGGTATTA |
| HEY1 | Forward | GCGTGGGAAAGGATGGTTGAG |
| | Reverse | CTCTCGGCTGCTTGCGTTC |
| JAG1 | Forward | GAGCTATTTGCCGACAAGGC |
| | Reverse | GGAGTTTGCAAGACCCATGC |
| miRNA-25 | | CATTGCACTTGTCTCGGT |
| MMP1 | Forward | CTGAAGGTGATGAAGCAGCC |
| | Reverse | AGTCCAAGAGAATGGCCGAG |
| MMP2 | Forward | GCGACAAGAAGTATGGCTTC |
| | Reverse | TGCCAAGGTCAATGTCAGGA |
| NOTCH1 | Forward | CCACCTCGTCTCTCCCACCT |
| | Reverse | ACAGCCACTCGCATTGACCATT |
| NOTCH3 | Forward | ACACCAATGCCCAGGACCAC |
| | Reverse | ATCAGTGCCGTTGAGCCATCTG |
| TGF-β1 | Forward | TGGAAACCCACAACGAAATC |
| | Reverse | GGGTTCAGGTACCGOTTCTC |
| TGF-β2 | Forward | GGATGCGGCCTATTGCTTTAGA |
| | Reverse | TCGTGTATCCATTTCCACCCTAGA |
| TGF-βRI | Forward | GCCTTGGTCCTGTGGAACTG |
| | Reverse | GGTCCTCTTCATTTGGCACTCG |
| TGF-βRII | Forward | GGCTCAACCACCAGGGCATCCAGAT |
| | Reverse | CTCCCCGAGAGCCTGTCCAGATGCT |
| TGF-βRIII | Forward | ACCGTGATGGGCATTGCGTTTGCA |
| | Reverse | GTGCTCTGCGTGCTGCCGATGCTGT |
| TIMP1 | Forward | TTCCGACCTCGTCATCAGGG |
| | Reverse | ATTCAGGCTATCTGGGACCGC |
| TIMP3 | Forward | ACCTGCCTTGCTTTGTGACTTCC |
| | Reverse | GGCGTAGTGTTTGGACTGGTAGC |

Commercial Primer

| Human Gene | Brand | Catalogue ID |
|---|---|---|
| RNU6-2 | Qiagen | 218300 |

### 6.4 TGF-β Stimulation Assay

24 h after cell transfection using Combination 3 (5, 20, and 40 pmol/mL), commercial miR-25 or negative control mimetics (20 pmol/mL), cells were stimulated with TGF-3 (10 ng/mL in DMEM) or control (RNase-free water). Cells were harvested a further 24 h later and the relative expression of COL1A1 and ACTA2 mRNA was analysed using qRT-PCR as described above. 6.5 SDS-PAGE and Western blot

Protein lysates were quantified using BCA assay (ThermoFisher Scientific). 10 μg of protein per sample was separated using SDS polyacrylamide gel electrophoresis (SDS-PAGE) followed by wet transfer to a low fluorescent PVFC membrane (Merck Millipore, Burlington, MA, USA) at 90V for 2 h. Membranes were blocked in Odyssey® TBS blocking buffer for 1 h at room temperature before incubated overnight at 40C with primary antibodies in Odyssey® TBS blocking buffer with 0.2% Tween-20 (antibodies and dilutions are listed in Table 5). Membranes were then washed and incubated with IRDye secondary antibodies (Table 6) in Odyssey® TBS blocking buffer, with 0.2% Tween-20 and 0.01% SDS for 1 h at room temperature. Membranes were scanned using the Odyssey® CLX infrared imaging system (Li-Cor Biosciences, Nebraska, USA) and analysed with Image Studio Lite Software version 5.2.5 (Li-Cor Biosciences). Protein expression of target genes was normalised to relative protein expression of β-actin.

TABLE 6
PRIMARY AND SECONDARY ANTIBODIES
USED FOR WESTERN BLOT ANALYSES

| | | | | | | | |
|---|---|---|---|---|---|---|---|
| | Predicted | | | | | | |
| | Band | | | | | | |
| | Size | | | | | | Storage |
| Protein | (kDa) | Species | Reactivity | Company | ID | Dilution | (° C.) |

Primary Antibodies

| | | | | | | | |
|---|---|---|---|---|---|---|---|
| ADAM-17 | 93 | Rabbit | Hu, Ms, Ra | Abcam | Ab39162 | 1:1000 | −20 |
| beta-Actin | 42 | mouse | Hu, Ms, Ra | Sigma | A5441 | 1:10000 | −20° C. |
| Collagen I | 130 | rabbit | Hu, Ms, Ra, | Abcam | ab34710 | 1:1000 | −20° C. |
| | | | Sh, Go, Ho, | | | | |
| | | | Co, Pi, | | | | |
| | | | Marmoset | | | | |
| FKBP14 | 24 | rabbit | Hu, Ms, | Abcam | ab251703 | 1:500 | −20° C. |
| | | | Orangutan | | | | |
| MMP2 | 74 | rabbit | Hu, Ms | Abcam | ab97779 | 1:1000 | −20° C. |
| TGF-beta | 56 | rabbit | Hu, Ms, Ra, | Abcam | ab31013 | 1:1000 | −20° C. |
| Receptor I | | | Co | | | | |

Secondary Antibodies

| | Wavelength | | | | | | |
|---|---|---|---|---|---|---|---|
| Antibody | (nm) | Species | Reactivity | Company | ID | Dilution | Storage |
| IRDye | 800CW | Donkey | Rabbit | LI-COR | Ab621848 | 1:20000 | 4° C. |
| Donkey | | | IgG | Biosciences | | | |
| anti- | | | | | | | |
| Rabbit | | | | | | | |
| IgG | | | | | | | |
| antibody | | | | | | | |
| IRDye | 680CW | Donkey | Mouse | LI-COR | Ab10953628 | 1:20000 | 4° C. |
| Donkey | | | IgG | Biosciences | | | |
| anti- | | | | | | | |
| Mouse | | | | | | | |
| IgG | | | | | | | |
| antibody | | | | | | | |

Abbreviations: Hu = human; Ms = mouse; Ra = rat; Sh = sheep; Go = goat; Ho = horse; Co = cow; Pi = pig.

### 6.6 Enzyme-Linked Immunosorbent Assay (ELISA)

LX-2 cells were seeded and transfected in 10 cm dishes with proprietary mimetic C3 or commercial miR-25 mimetic (20 pmol/mL and 40 pmol/mL), or negative control mimetic (40 pmol/mL), as described above. After 24 h, cells were washed with 1×PBS and media was replaced with fresh DMEM. 48 h after transfection, cell culture media was collected and centrifuged at 2,000×g for 10 minutes to remove debris. Supernatant was stored at −80° C. Cells were washed again and cell media was replaced. 72 h after transfection, cell culture media was collected as previously and stored at −80° C. To analyse changes in COL1A1 secretion following miR-25 transfection, the Human Pro-Collagen I alpha 1 SimpleStep ELISA® Kit (Abcam, Cambridge, United Kingdom) was used. Samples were diluted 1:64 and analysed according to the manufacturer's protocol.

### 6.7 Cell Migration Analysis

24 hours after transfection of LX-2 cells with proprietary mimetic C3 or commercial miR-25 mimetic (20 pmol/mL and 40 pmol/mL), or negative control mimetic (40 pmol/mL), cells were reseeded into 96-well cell culture plates with 4.5×10⁴ cells/well. 24 h later, once cells were confluent, a wound was implemented using a scratch wound maker (Essen Bioscience, Ann Arbor, Michigan, USA) and plates were incubated in the IncuCyte Zoom live cell analysis system (Essen Bioscience) for 24 h. Wound closure over time was measured in terms of wound width (μm). Data was normalised in GraphPad Prism (version 8.4.3; GraphPad Software, San Diego, CA, USA).

### 6.8 Cell Proliferation Assay

A cell proliferation assay to analyse changes in HSC proliferation was performed as previously described. LX-2 cells were transfected with proprietary mimetic C3 or commercial miR-25 (20 pmol/mL and 40 pmol/mL), or negative control mimetics (40 pmol/mL). After 24 hours, transfected cells were reseeded in triplicate in 12-well plates with 2×10⁵ cells/well, and plates were incubated in the IncuCyte Zoom live cell analysis system (Essen Bioscience) for up to 7 days. Cell proliferation was measured in terms of cell confluence (%) over time. Data was normalised to baseline in GraphPad Prism and a one-phase association non-linear regression was performed to calculate the growth constant (K) as an indication of cell proliferation rate.

### 6.9 Cell Contractility Assay

A collagen contraction assay to analyse changes in HSC contractility was performed as previously described. LX-2 cells were transfected with proprietary mimetic C3 or commercial miR-25 (20 pmol/mL and 40 pmol/mL), or negative control mimetics (40 pmol/mL). Type 1 collagen solution from bovine skin (Sigma) was adjusted to physiological pH and stiffness, following manufacturer's instructions. In 24-well cell culture plates, 900 μl/well was allowed to set over night at 37° C. to form a collagen lattice. 24 hours after transfection, cells were reseeded with 1×10⁵ cells/well onto the collagen lattice in 1 mL of serum-free DMEM (1% glutamine, and 1% penicillin/streptomycin). A further 24 hours later, cell contraction was stimulated by adding endothelin-1 (10 nM, Sigma) and photos of the culture plate were taken with a gel imager (Vilber Lourmat, Collégien, France) at 0.5, 1, 2.5 and 6 h. Collagen matrix shrinkage over time was measured in ImageJ software (version 1.5j8, National Institute of Health, USA). Data was normalised in GraphPad Prism.

### 6.10 Statistical Analyses

All data is expressed as mean±standard error of the mean (SEM). GraphPad Prism (version 8.4.3) was used for statistical analyses of data. Data was tested for normality using the Shapiro-Wilk normality test. All results were analysed using one-way ANOVA with Dunnett's multiple comparisons post-hoc analysis. Significant differences were defined as P<0.05.

The disclosure of every patent, patent application, and publication cited herein is hereby incorporated herein by reference in its entirety.

The citation of any reference herein should not be construed as an admission that such reference is available as “prior art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Geerts, A. History, heterogeneity, developmental biology, and functions of quiescent hepatic stellate cells. Semin Liver Dis 21, 311-335, doi:10.1055/s-2001-17550 (2001).
Friedman, S. L. Hepatic Stellate Cells: Protean, Multifunctional, and Enigmatic Cells of the Liver. Physiological Reviews 88, 125-172, doi:1 0.1152/physrev.00013.2007 (2008).
Bataller, R. & Brenner, D. A. Liver fibrosis. J Clin Invest 115, 209-218, doi:10.1172/JC124282 (2005).
Mederacke, I. et al. Fate tracing reveals hepatic stellate cells as dominant contributors to liver fibrosis independent of its aetiology. Nature Communications 4, 2823, doi:1 0.1 038/ncomms3823 (2013).
Dooley, S. et al. Transforming growth factor beta signal transduction in hepatic stellate cells via Smad2/3 phosphorylation, a pathway that is abrogated during in vitro progression to myofibroblasts. TGFbeta signal transduction during transdifferentiation of hepatic stellate cells. FEBS Lett 502, 4-10 (2001).
Villanueva, A. et al. Notch signaling is activated in human hepatocellular carcinoma and induces tumor formation in mice. Gastroenterology 143, 1660-1669.e1667, doi:10.1 053/j.gastro.2012.09.002 (2012).
Xie, G. et a/. Cross-talk between Notch and Hedgehog regulates hepatic stellate cell fate in mice. Hepatology (Baltimore, Md.) 58, 1801-1813, doi:10.1002/hep.26511 (2013).
Bansal, R., van Baarlen, J., Storm, G. & Prakash, J. The interplay of the Notch signaling in hepatic stellate cells and macrophages determines the fate of liver fibrogenesis. Scientific Reports 5, 18272, doi:10.1038/srepl8272 (2015).
Wang, Y. et a/. Notch signaling mediated by TGF-β/Smad pathway in concanavalin A-induced liver fibrosis in rats. World J Gastroenterol 23, 2330-2336, doi:10.3748/wjg.v23.i13.2330 (2017).
Ie Sage, C. et al. Regulation of the p27(Kip1) tumor suppressor by miR-221 and miR-222 promotes cancer cell proliferation. EMBO J 26, 3699-3708, doi:10.1038/sj.emboj.7601790 (2007).
Yu, F. et al. let-7 Regulates Self Renewal and Tumorigenicity of Breast Cancer Cells. Cell 131, 1109-1123, doi:10.1016/j.cell.2007.10.054 (2008).
Eichhorn, S. W. et al. mRNA destabilization is the dominant effect of mammalian microRNAs by the time substantial repression ensues. Mol Cell 56, 104-115, doi:10.1016/j.molcel.2014.08.028 (2014).
Guo, H., Ingolia, N. T., Weissman, J. S. & Bartel, D. P. Mammalian microRNAs predominantly act to decrease target mRNA levels. Nature 466, 835-840, doi:10.1038/nature09267 (2010).
Bartel, D. P. MicroRNAs: Target Recognition and Regulatory Functions. Cell 136, 215-233, doi:https://doi.org/10.1016/j.cell.2009.01.002 (2009).
Ha, M. & Kim, V. N. Regulation of microRNA biogenesis. Nature Reviews Molecular Cell Biology 15, 509-524, doi:10.1038/nrm3838 (2014).
Krauskopf, J. et a/. Serum microRNA signatures as “liquid biopsies” for interrogating hepatotoxic mechanisms and liver pathogenesis in human. PLoS One 12, e0177928-e0177928, doi:10.1371/journal.pone.0177928 (2017).
Ichimura, A., Ruike, Y., Terasawa, K. & Tsujimoto, G. miRNAs and regulation of cell signaling. The FEBS Journal 278, 1610-1618, doi:10.1111/j.1742-4658.2011.08087.x (2011).
Inui, M., Martello, G. & Piccolo, S. MicroRNA control of signal transduction. Nature Reviews Molecular Cell Biology 11, 252-263, doi:10.1038/nrm2868 (2010).
Cook, N. L., Pereira, T. N., Lewindon, P. J., Shepherd, R. W. & Ramm, G. A. Circulating microRNAs as noninvasive diagnostic biomarkers of liver disease in children with cystic fibrosis. J Pediatr Gastroenterol Nutr 60, 247-254, doi:10.1 097/MPG.0000000000000600 (2015).
Genz, B. et al. Overexpression of miR-25-3p inhibits Notch1 signaling and TGF-β-induced collagen expression in hepatic stellate cells. Scientific Reports 9, 8541, doi:1 0.1 038/s41598-01 9-44865-1 (2019).

What is claimed is:

1. A composition comprising a miR-25 mimetic compound, wherein the miR-25 mimetic compound comprises:
a. a first strand comprising a nucleotide sequence that corresponds to residues 52-73 of the mature miR-25 sequence set forth in SEQ ID NO:1 conjugated to two uracil resides at the 3′ termini, and having at least one modified nucleotide; and
b. a second strand that hybridises at least under low stringency conditions to the first strand and having at least one modified nucleotide.

2. The composition of claim 1, wherein the modified nucleotides comprise nucleotides with backbone modifications and/or modified sugar residues.

3. The composition of claim 2, wherein the backbone modification comprises one or more phosphorothioate, morpholino, or phosphonocarboxylate linkages.

4. The composition of claim 3, wherein the first two nucleotides at the 5′ termini of the first strand are linked to each other by phosphorothioate linkages.

5. The composition of claim 3 or claim 4, wherein the last 4 or 5 nucleotides of the 3′ termini of the first strand are linked to each other by phophorothioate linkages.

6. The composition of any one of claims 1 to 5, wherein at least one of the 3′ nucleotides of the first strand is a 2′-O-methyl modified nucleotide.

7. The composition of any one of claims 1 to 6, wherein at least one nucleotide of the first strand is a 2′-fluoro nucleotide.

8. The composition of any one of claims 1 to 7, wherein the first strand does not have a modified sugar residue at the second position at both the 5′ and 3′ terminis.

9. The composition of any one of claims 1 to 8, wherein at the backbone modification of the second strand comprises one or more of a phosphorothioate, morpholino, or phosphonocarboxylate linkage.

10. The composition of claim 9, wherein the first two nucleotides of the 5′ termini of the second strand are linked by a phosphorothioate linkage.

11. The composition of claim 9 or claim 10, wherein the last seven nucleotides of the 3′ termini of the second strand are linked by phosphorothioate linkages.

12. The composition of any one of claims 1 to 11, wherein the first nucleotide at the 5′ termini of the second strand is a 2′-O-methyl modified nucleotide.

13. The composition of any one of claims 1 to 11, wherein the first seven nucleotides at the 3′ termini of the second strand are 2′-O-methyl modified nucleotides.

14. The composition of any one of claims 1 to 13, wherein the miR-25 mimetic compound is presented in Table 1:
TABLE 1

| | |
|---|---|
| | SEQ |
| | ID |
| Sequence (5′ to 3′) | NO: |

Second/sense/passenger strand

| | |
|---|---|
| 5′-mU*rC.rA.rG.rA.rC.rC.rG.rA.rG.rA.rC. | 5 |
| rA.rA.rG.rU.rG.mC*mA*mA*mU*mG*mU*mU-3′ | |

First/antisense/guide strands

| | |
|---|---|
| 5′-rC*rA.rU.rU.rG.rC.rA.rC.rU.rU.rG.rU. | 2 |
| rC.rU.rC.rG.rG.rU.rC.rU*rG*rA*rU*rU-3′ | |
| 5′-rC*rA.rU.rU.rG.rC.rA.rC.rU.rU.rG.rU. | 14 |
| rC.rU.rC.rG.rG.mU.rC.mU.rG*rA*rU*rU-3′ | |
| 5′-rC*rA.fU.rU.rG.fC.rA.fC.rU.rU.rG.rU. | 16 |
| rC.rU.rC.rG.rG.mU.rC.mU.rG*rA*rU*rU-3′ | |

TABLE 2

| | |
|---|---|
| | SEQ |
| | ID |
| Sequence (5′ to 3′) | NO: |

Second/sense/passenger strand

| 5′-mU*rC.rA.rG.rA.rC.rC.rG.rA.rG.rA.rC. | 5 |
|---|---|
| rA.rA.rG.rU.rG.mC*mA*mA*mU*mG*mU*mU-3′ | |
| First/antisense/guide strands | |
| 5′-rC*rA.rU.rU.rG.rC.rA.rC.rU.rU.rG.rU. | 4 |
| rC.rU.rC.rG.rG.mU.mC.mU*mG*mA*mU*mU-3′ | |
| 5′-rC*mA.rU.rU.rG.rC.rA.rC.rU.rU.rG.rU. | 11 |
| rC.rU.rC.rG.rG.rU.rC.rU*rG*rA*rU*rU-3′ | |
| 5′-rC*rA.rU.rU.rG.rC.rA.rC.rU.rU.rG.rU. | 12 |
| rC.rU.rC.rG.rG.rU.rC.rU*rG*rA*mU*rU-3′ | |
| 5′-rC*rA.fU.rU.rG.fC.rA.fC.rU.rU.rG.rU. | 15 |
| rC.rU.rC.rG.rG.rU.rC.rU*rG*rA*rU*rU-3′ | |
| 5′-rC*rA.fU.rU.rG.fC.rA.fC.rU.rU.rG.rU. | 17 |
| rC.rU.rC.rG.rG.fU.rC.fU*rG*rA*rU*rU-3′ | |

15. The composition of any one of claims 1 to 14, wherein the first strand comprises a sequence selected from SEQ ID NO: 2, 4, 9, 11, 12, 13, or 14 and the second strand comprises the sequence set forth in of SEQ ID NO:5.

16. The composition of any one of claims 1 to 15, wherein the first strand comprises the sequence of SEQ ID NO:2 and the second strand comprises the sequence of SEQ ID NO:5.

17. The composition of any one of claims 1 to 15, wherein the first strand comprises the sequence of SEQ ID NO:14 and the second strand comprises the sequence of SEQ ID NO:5.

18. The composition of any one of claims 1 to 15, wherein the first strand comprises the sequence of SEQ ID NO:16 and the second strand comprises the sequence of SEQ ID NO:5.

19. A pharmaceutical composition comprising the composition of any one of claims 1 to 18, together with a pharmaceutically acceptable carrier, excipient or diluent.

20. A method for treating fibrosis in a subject, comprising administering to the subject the pharmaceutical composition of claim 19.

21. A method for treating fibrosis in a subject, wherein the fibrosis is liver fibrosis.

22. The method of claim 20 or claim 21, wherein the expression of COL1A1, COL1A2, COL3A1, COL4A3, COL5A2, COL11A1, FN1, MMP2, CTGF, TGFB2, and/or TGFB3 is reduced.

23. The method of any one of claims 20 to 22, wherein the miR-25 mimetic compound is presented in Table 1 or Table 2.

24. The method of any one of claims 20 to 22, wherein the first strand comprises a sequence selected from SEQ ID NO: 2, 4, 9, 11, 12, 13, or 14, and/or the second strand comprises a sequence selected from any one of SEQ ID NOs:5.

25. The method of any one of claims 20 to 24, wherein the first strand comprises the sequence of SEQ ID NO:2 and the second strand comprises the sequence of SEQ ID NO:5.

26. The method of any one of claims 20 to 25, wherein the first strand comprises the sequence of SEQ ID NO:11 and the second strand comprises the sequence of SEQ ID NO:5.

27. The method of any one of claims 20 to 26, wherein the first strand comprises the sequence of SEQ ID NO:13 and the second strand comprises the sequence of SEQ ID NO:5.

28. The method of any one of claim 20 to 27, wherein the subject is a human.
